(19) United States
(12) Patent Application Publication
(10) Pub. No.: US 2007/0282537 A1
(43) Pub. Date: Dec. 6, 2007

(54) RAPID CHARACTERIZATION OF POST-TRANSLATIONALLY MODIFIED PROTEINS FROM TANDEM MASS SPECTRA

(75) Inventors: Michael A. Freitas, Columbus, OH (US); Hua Xu, Columbus, OH (US)

Correspondence Address: DINSMORE & SHOHL LLP, ONE DAYTON CENTRE, ONE SOUTH MAIN STREET, SUITE 1300, DAYTON, OH, 45402-2023, US

(73) Assignee: THE OHIO STATE UNIVERSITY, Columbus, OH (US)

(21) Appl. No.: 11/754,700

(22) Filed: May 29, 2007

Related U.S. Application Data
(60) Provisional application No. 60/809,047, filed on May 26, 2006.

(51) Int. Cl. G06F 19/00; G01N 33/00

(52) U.S. Cl. 702/19; 702/1; 702/20

(57) ABSTRACT

A software algorithm that matches tandem mass spectra created simultaneously and automatically to theoretical peptide sequences derived from a protein database is disclosed. The program characterizes shotgun proteomic data sets obtained from proteins (such as histones) that possess extensive posttranslational modifications that are often difficult to characterize. Data is searched against all theoretical peptides including all combinations of modifications. The program returns four scores to assess the quality of match. The employed algorithm is sensitive to mass accuracy. For high mass accuracy data, a false positive rate as low as 2% may be achieved. Monte Carlo Simulations were also used to obtain a solution to statistical models and calculate statistical scores. The program can also be used to automatically and directly identify disulfide linked proteins and peptides in tandem mass spectra without chemical reduction and/or other derivatization using a probabilistic scoring model.

### BACKGROUND OF THE INVENTION

The present invention generally relates to a tandem mass spectrometry database search program and, in particular, relates to a tandem mass spectrometry database search program that matches tandem mass spectra created automatically and simultaneously to theoretical peptides derived from a protein database.

Mass spectrometry (MS) is an analytical technique used to measure the mass-to-charge (m/z) ration of ions. Database searching in combination with shotgun proteomics is the major tool used to identify peptides and proteins in complex protein mixtures. Database searching programs match experimental spectra with theoretical spectra created from the database. They are classified into four categories according to their scoring algorithms: descriptive, interpretative, stochastic and statistical/probabilistic. SEQUEST is an example of a descriptive model and one of the most commonly used database searching programs. Other programs of this type include Sonar and SALSA. PeptideSearch is based on an interpretative model for database searching. SCOPE and OLAV use stochastic models for database searching. A group of programs that are based on statistical and probabilistic models have also been developed. Among them, Mascot is the most commonly used. The probability based score is a direct measure of the probability that the match is significant. Probability based scores from different search algorithms can be directly compared, whereas descriptive score must be converted to probabilities for comparisons.

These programs score candidate matches for the experiment spectra with theoretically generated counterparts from a protein database. Low mass accuracy, noise and low signal to noise ratio can compromise search results from database searching programs. There are inconsistencies between searching results from different searching programs due to their different scoring algorithms. Kapp et al performed a systematic comparison of various database searching algorithms including Mascot, SEQUEST, Sonar, Spectrum Mill and X!Tandem. A data set containing 3952 tandem MS spectra created from human B3-Cit-plasma (Asian-American) on an LCQ Deca XP ion trap mass spectrometer was searched using all five algorithms. The trypsin-constrained search results from the five algorithms achieved consensus for 345 spectral matches. Considering all search results the algorithms matched 662 spectra.

Additionally, liquid chromatography coupled with tandem mass spectrometry (LC-MS/MS) has become one of the most used tools in mass spectrometry based proteomics. In shotgun proteomics, peptides are separated using liquid chromatography and introduced into a mass spectrometer via an ionization interface. In tandem mass spectrometry, the peptide precursor ions are isolated and fragmented via collision-induced dissociation (CID) with inert gas, electron capture dissociation (ECD), surface induced dissociation (SID) and/or electron transfer dissociation (ETD). The resulting tandem MS spectra contain product ion signatures that relate back to the identity of the peptide precursor ions.

It has been reported that improving mass accuracy of precursor and product ions produced during data-dependent LC-MS/MS can significantly improve the confidence of identification of peptides and lower the rates of false and ambiguous identifications. More instruments capable of performing LC-MS/MS with high mass resolution and high mass accuracy are becoming available to the research community. High mass accuracy can be used to filter spectra whose precursor ions fall outside the mass accuracy resulting in fewer spectral comparisons. Similarly searches using high mass accuracy product ion spectra reduce the likelihood that the theoretical spectra can randomly match the experimental. Either of these approaches or their combination will result in a reduction of false positive matches. While some algorithms take advantage of mass accuracy, the full potential of mass accuracy has not been fully exploited.

Theses various algorithms have been developed to automate the process for modern high-throughput LC-MS/MS experiments. These algorithms fall under two categories: de novo sequence inference and database searching. The first approach identifies peptide sequences directly from the tandem MS data. This type of algorithm is usually computationally expensive and limited by the mass accuracy of the tandem MS data. The database searching algorithms identify peptides by comparison with a protein sequence database. In this approach, all potential peptides are created from the sequence database via digestion with proteases. Theoretical spectra containing product ion series appropriate for the given fragmentation technique are created for the peptides. All tandem MS spectra in the data set are then compared with the theoretical spectra. Because of their relatively lower computation expense and higher compatibility with low mass accuracy spectra, database searching programs are more commonly used at this time.

There are also various probability based post-search methods used to statistically curate search results from database search algorithms. These methods estimate the accuracy of protein/peptide identifications and compare search results from different algorithms based on a common standard. However, many models involve empirical parameters such as score from correlative scoring algorithms. Therefore they may possess biases as a result of parameter optimization or model training. The key comparison between different algorithms lies in how each approach scores a potential match between experimental and theoretical spectra.

Therefore, there is a need for a new search program that uses a mass accuracy sensitive probabilistic scoring algorithm that lowers false positive rate and improves confidence in peptide assignment and protein identification. This algorithm is separate and distinct from algorithms that filter matches based on mass accuracy. In the latter high mass accuracy can be used to filter spectra by only searching tandem mass spectra whose precursor ion falls within the stated mass tolerance, and filtering product ions by high mass accuracy can further reduce the likelihood of a random match. However, a score sensitive model implicitly takes mass accuracy into account during scoring.

There is another need for a novel statistically derived algorithm to rigorously calculate protein scores from the statistically based peptide scores. Thus the protein scores reflect the significance of protein hits and can be used to differentiate true protein hits from random ones.

There is yet another need for a proteomics tool that matches experimental tandem mass spectra created on-the-fly to theoretical peptide sequences derived from a protein sequence databases to catch all possible peptide hits and make data searching time shorter than data acquisition time.

Database search programs are widely used to identify and characterize proteins, peptides and their post-translational modifications from resulting tandem spectrometric data. The database search process involves creation of all proteolytic peptides contained within a protein sequence database. Theoretical tandem mass spectra for these peptides are then generated based on the fragmentation technique used. Experimental spectra are then compared with the theoretical ones created from the sequence database. Several database search programs have been developed to automate this process. These programs rely on different scoring models to evaluate potential peptide matches for experimental spectra and differentiate true peptide identifications from false ones. There are four types of scoring models that are currently used in these database search programs: descriptive, interpretative, stochastic and statistical/probabilistic models. SEQUEST was the first automated approach and is still one of the most commonly used database search programs that use a descriptive scoring model. PeptideSearch and GutenTag are examples of database search programs that employ an interpretative scoring model. Database search programs that use stochastic models include SCOPE and OLAV. Among all the programs with statistical/probabilistic scoring models, Mascot is most commonly used.

The statistical/probabilistic scoring models have gained increasing interests, since probability-based scores are a direct measure of the statistical confidence of a peptide match and scores from different models can be directly compared. However, most of the statistical scoring algorithms ignore the information about the sequence tags of the peptides inferred from the tandem mass spectra and/or the information of abundances of peaks in the experimental data. Abundance and sequence tag based scoring models used in database search are normally very complex. The abundance and sequence tag based scoring models that have been reported are either empirical models or models with empirically estimated parameters or approximations.

Therefore, there is still yet another need for a new approach based on random sampling to perform abundance and sequence tag based statistical scoring of peptide matches in database search of tandem mass spectrometric data. The approach applies Monte Carlo Simulations (MCS) to obtain a solution to the statistical models and calculate statistical scores. Monte Carlo methods are especially useful in solving complex statistical models that cannot be solved without making assumptions or simplifications, such as scoring models in database search programs involving many variables or variables with unknown distributions (such as product ion abundances and sequence tags). By this approach, complex scoring models may be implemented in database search software to take into account as much bioinformatics information as possible but yet remain solvable and provide statistical scores without introducing additional assumptions or simplifications. However, Monte Carlo methods normally suffer high computational expense and the values are estimated with a variance that is influenced by sampling size. Therefore, in order to make the methods practical for use in high-throughput database search programs, the computational algorithms for the methods need to be optimized for speed and must employ variance reduction techniques to obtain reasonable estimation precision.

In addition, among the 20 natural amino acids, cysteine is unique because it involved in many biological activities through oxidation and reduction to form disulfide bonds and sulfhydryls. These modifications play an important role in protein's structure and biological function. The identification and characterization of protein disulfide bonds is an essential step to thoroughly understand their biological function. However, the determination of disulfide linkages can be challenging.

Several methods are used to determine the pattern of disulfide bond linkage. Among them, crystallography and nuclear magnetic resonance (NMR) are excellent tools that can identify disulfide bond linkages with minimal disulfide bond inter-exchange. However, the application of both techniques is limited by large sample requirements and protein size. Edman Degradation can also be used for the identification of disulfide bonds. But this technique requires ultra pure samples, digestion and further purification of the peptides for protein sequencing. Mass spectrometry (MS) has also been successfully applied to identify disulfide bonds in solution and from gels. Researchers have successfully demonstrated that disulfide bridge patterns can be identified by MS analysis, following protein digestion either under partial reduction or non-reduction conditions.

Partial reduction is a widely accepted approach for the determination of disulfide bonds. In this approach, the protein is digested under controlled conditions such that disulfide bonds with different reduction kinetics are reduced and alkylated gradually. Peptide containing modified cysteines are further separated and analyzed by MS and/or tandem MS. However, when a protein is highly bridged, multiple reductions and separations are necessary for complete disulfide mapping. The approach requires relatively large sample amounts compared with direct tandem MS and can provide ambiguous results when disulfide bonds have the same or similar reduction rates. Tandem MS of protein digests under non-reducing condition has also been used frequently for the investigation of disulfide bond patterns. With appropriate enzyme(s) digestion, disulfide bonds pattern can be identified in a single run. However, data processing may be extremely complicated and time/labor-consuming for proteins with multiple unknown disulfide bonds. Also, when the sample contains large number of cysteines or multiple disulfide bonds exist in the sample, some disulfide bond linkages might go unidentified. Tools that can effectively search tandem MS data for the presence of disulfide bonds will facilitate disulfide analysis.

Several methods have been reported for peptide mass analysis for disulfide-linked peptides. Tandem MS data from disulfide-linked peptides under reduction conditions can be analyzed using traditional database search programs. However, automated high-throughput software for analysis of tandem MS data of disulfide-linked proteins and peptides under non-reducing condition are limited. Recently, a search program for the identification of disulfide bonds in peptides from tandem MS data was reported by Wefing et al. However, the program lacks a calibrated empirical scoring model or a statistical scoring model. Commonly used database search programs, such as SEQUEST and Mascot, do not have options to perform automated analysis of tandem MS data from disulfide-linked proteins and peptides.

Therefore, there is a need for an automated data search algorithm for tandem MS data to identify disulfide bonds in proteins and peptides.

### BRIEF SUMMARY OF THE INVENTION

According to the present invention, a program that matches tandem mass spectra created automatically and simultaneously to theoretical peptide sequences derived from a protein database is disclosed. The program was designed to characterize shotgun proteomic data sets obtained from proteins (such as histones) that possess extensive post-translational modifications (PTMs) that are often difficult to characterize. Data is searched against all theoretical peptides including all combinations of modifications. The program returns four scores to assess the likelihood and quality of the match. These scores comprise an empirically derived score and three statistical probabilities that calculate the likelihood that a hit could be random based separately on the number of matched peaks, the distribution of ion abundances and the number of sequence tags. After scoring all the matches, the program performs an additional statistical evaluation of the resulting peptide scores, excludes all insignificant matches based on the parameters specified by the users and calculates the likelihood that the protein matches could be random. Monte Carlo Simulations were used to obtain a solution to statistical models and calculate statistical scores.

The program is portable and can be run on personal computers (PCs) running either Linux or Microsoft Windows. A parallelized version is also available which can be run on distributed memory clusters. On modern PCs, the program can search against >10,000 peptide sequences per second whereas the parallel version scales linearly with number of CPUs and can search against >1,000,000 peptide sequences per second.

An automated data search algorithm for tandem MS data to identify disulfide bonds in proteins and peptides is also presented. The algorithm employs a probabilistic scoring model and fast database searching algorithm to achieve reliable statistical scoring, high sensitivity and selectivity and high performance identification of disulfide bonds from proteins and peptides. With this approach, disulfide bonds in proteins and peptides can be identified along with other fixed and/or variable modifications in tandem mass spectrometry and without the need for reduction or derivatization of the sulfhydryls and/or disulfide bonds.

In accordance with one embodiment of the present invention, the tandem mass spectrometry database search program searches the experimental data against all possible theoretical peptides including all combinations of modifications.

In accordance with another embodiment of the present invention, the tandem mass spectrometry database search program was developed that employs an algorithm sensitive to mass accuracy.

In accordance with yet another embodiment of the present invention, the highly parallelized version of the tandem mass spectrometry database search program allows for automated searches of large data sets against large databases including a large number of PTMs.

Accordingly, it is a feature of the embodiments of the present invention to have a tandem mass spectrometry database search program that employs an algorithm sensitive to mass accuracy with a low false positive rate and that allows for automated and simultaneous searches of large data sets against large databases.

It is another feature of the embodiments of the present invention to identify disulfide linked peptides without chemical reduction via tandem mass spectrometry. Other features of the embodiments of the present invention will be apparent in light of the description of the invention embodied herein.

### BRIEF DESCRIPTION OF THE SEVERAL VIEWS OF THE DRAWINGS

The following detailed description of specific embodiments of the present invention can be best understood when read in conjunction with the following drawings, where like structure is indicated with like reference numerals and in which:

FIG. 1 illustrates an example output from the tandem mass spectrometry database search program according to an embodiment of the present invention.

FIG. 2 illustrates estimation precise of various sampling sizes (a) abundance-based Monte Carlo Simulation (MCS) model and (b) sequence-based MCS model according to an embodiment of the present invention.

FIG. 3 is a flow diagram of MassMatrix database search algorithm that employs MCS-based scoring needs according to an embodiment of the present invention.

FIG. 4 illustrates Receiver Operating Characteristic (ROC) curves of different scoring standards for peptides according to an embodiment of the present invention.

FIG. 5 illustrates distributions of pp2_{MCS} scores from the MCS-based scoring models for peptide according to an embodiment of the present invention.

FIG. 6 illustrates distributions of pp_{tag} scores from the MCS-based scoring models for peptide according to an embodiment of the present invention.

FIG. 7 illustrates examples of spectral influences on scoring according to an embodiment of the present invention.

FIG. 8 illustrates the effect of mass accuracy on the theoretical probability density function (ƒ(pp)) of pp and pp2 values for random matches (β≧β_{c}) according to an embodiment of the present invention.

FIG. 9 is a flow chart of the overall scheme for the MassMatrix search algorithm according to an embodiment of the present invention.

FIG. 10 depicts a Venn Diagrams showing consensus of peptides matches from MassMatrix and Mascot and SEQUEST according to an embodiment of the present invention.

FIG. 11 illustrates ROC curves of MassMatrix, Mascot and SEQUEST for a Orbitrap data set according to an embodiment of the present invention.

FIG. 12 illustrates the distributions of pp scores at different mass accuracies according to an embodiment of the present invention.

FIG. 13 illustrates peptide match before and after noise filtering according to an embodiment of the present invention.

FIG. 14 illustrates score distribution of protein hits after searching against human database including decoy sequences according to an embodiment of the present invention.

FIG. 15 illustrates speedup for the message passing interface of the tandem mass spectrometry database search program on a parallelized computer version according to an embodiment of the present invention.

FIG. 16 is a flow chart of the overall scheme of the PC version of the tandem mass spectrometry database search program according to an embodiment of the present invention.

FIG. 17 is a flow chart of the overall scheme of full-scan searching of the tandem mass spectrometry database search program according to an embodiment of the present invention.

FIG. 18 illustrates digestion of the protein sequence with the tandem mass spectrometry database search program according to an embodiment of the present invention.

FIG. 19 illustrates the procedure for checking if the peptide had been searched before by the tandem mass spectrometry database search program according to an embodiment of the present invention.

FIG. 20 illustrates modification with the tandem mass spectrometry database search program according to an embodiment of the present invention.

FIG. 21 is a flow chart of the overall scheme of the tandem mass spectrometry database search program on a parallelized computer version according to an embodiment of the present invention.

FIG. 22 illustrates the binary MPI gathering algorithm of the tandem mass spectrometry database search program on a parallelized computer version according to an embodiment of the present invention.

FIG. 23 illustrates classification of disulfide-linked peptides in MassMatrix according to an embodiment of the present invention.

FIG. 24 is a flow diagram for the search algorithm of disulfide-linked peptides in MassMatrix according to an embodiment of the present invention.

FIGS. 25a-d illustrates disulfide-linked peptide matches for (a) type 1A, (b) type 2A, (c) type 2B and (d) type 3 peptides according to an embodiment of the present invention.

FIGS. 26a-b illustrates disulfide bonds mapped by tandem MS experiment and subsequent database search in MassMatrix for digests under (a) basic and (b) acidic conditions according to an embodiment of the present invention.

FIG. 27 illustrates the distribution of pp values for peptides without disulfide bonds and disulfide-linked peptides, compared with false positive peptide matches according to an embodiment of the present invention.

FIG. 28 illustrates ROC curves of the search with decoy sequences. -: true positive peptide matches with disulfide bonds vs. false positives; --: true positive peptides without disulfide bonds vs. false positives according to an embodiment of the present invention.

### DETAILED DESCRIPTION

In the following detailed description of the embodiments, reference is made to the accompanying drawings that form a part hereof, and in which are shown by way of illustration, and not by way of limitation, specific embodiments in which the invention may be practiced. It is to be understood that other embodiments may be utilized and that logical, mechanical and electrical changes may be made without departing from the spirit and scope of the present invention.

Theoretical spectra for each putative proteolytic peptide sequence are created on-the-fly and matched against the experimental data. The tandem mass spectrometry database search program searches all possible peptides created from the selected protein database. A matrix-based searching algorithm is employed to accelerate the searching. Three scores are used to evaluate each match. These scores consist of an empirically derived score and two statistical probabilities that calculate the random likelihood of a match. After scoring all the matches, the software performs an additional statistical evaluation of the resulting peptide scores, excludes all insignificant matches based on the parameters specified by the users and calculates the random likelihood for the protein matches. Protein scores are calculated by the same statistical model used to calculate peptide scores. Additional features include reporting the sequence tag coverage for the peptides and proteins and color coding the sequences to aid in rapid visual interpretation. The software outputs results in HTML. The MPI (Message Passing Interface) library was used to parallelize the program on distributed memory clusters.

A matrix based computational model is used to perform digestion, modification and fragmentation processes in the search. All theoretical spectra is calculated on the fly, i.e., no pre-computed reference database is used, thereby making searching all combinations of modified and unmodified peptides possible. It is also possible to search all kinds of user-defined searching methods and parameters, such as, for example, proteolysis reagents, modifications, protein sequence databases and the like.

The performance of the tandem mass spectrometry database search program was evaluated using two tandem mass spectrometry (MS) data sets of human histones. The data sets contain tandem mass spectrometry (MS/MS) spectra obtained on an LTQ-Orbitrap hybrid mass spectrometer. The LTQ MS/MS data set was obtained with a mass accuracy of ˜1.0 Da and the Orbitrap with a mass accuracy of ˜0.01 Da. The data set with low accuracy is denoted as LTQ data set and the high accuracy set is denoted as Orbitrap data set.

The two data sets were searched with modifications of acetylation of lysine, acetylation of N-terminus, methylation of lysine, methylation of arginine and oxidation of methionine against the NCBlnr human database and a limited human histone database using the MS/MS database search program. Peptide sequences with a length from 4 to 30 amino acid residues, missed cleavage site of up to 3, and charges of +1, +2 and +3 were searched. Only b and y ions were considered in the fragmentation model. The maximum charge of product ions was set to +2. A dynamic noise level (DNL) algorithm was also employed to filter noise peaks in experimental spectra that cover over 10% of the detection range.

Additionally, the MS/MS database search program automates database searching of tandem MS data against sequence database with cistine-cistine bonds, branched protein sequences and modification groups that undergo fragmentation during peptide ion fragmentation process by considering six categories of peptides with cistine-cistine bonds created from protein sequence database. A searching algorithm that can search peptide with multiple chains is used to search peptides with cistine-cistine bonds, branches and modification groups that undergo fragmentation.

The two data sets were also evaluated using a competitor's program, Mascot, from Matrix Science Inc. The search parameters in Mascot were the same as in the MS/MS database search program. The two data sets were also searched against decoy databases with reversed and randomized protein sequences to compare the selectivity. Reversed human database was created by reversing all the sequences in the original human database. Randomized human database was created by reordering all the amino acid residues in random order for each protein sequence in the human database.

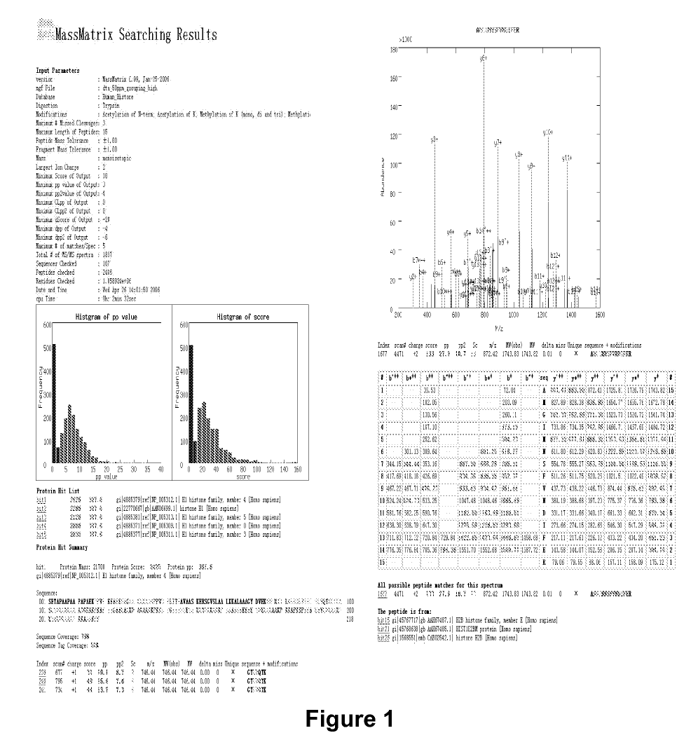

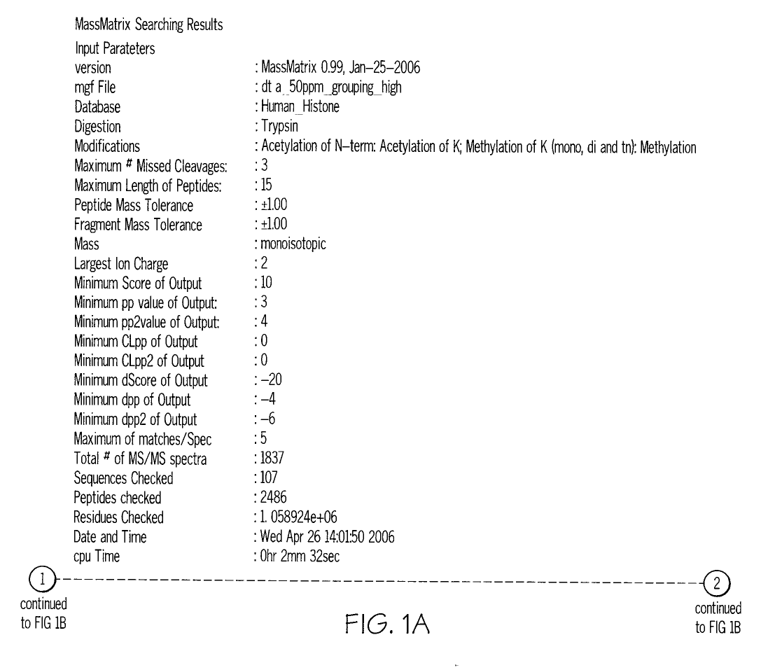

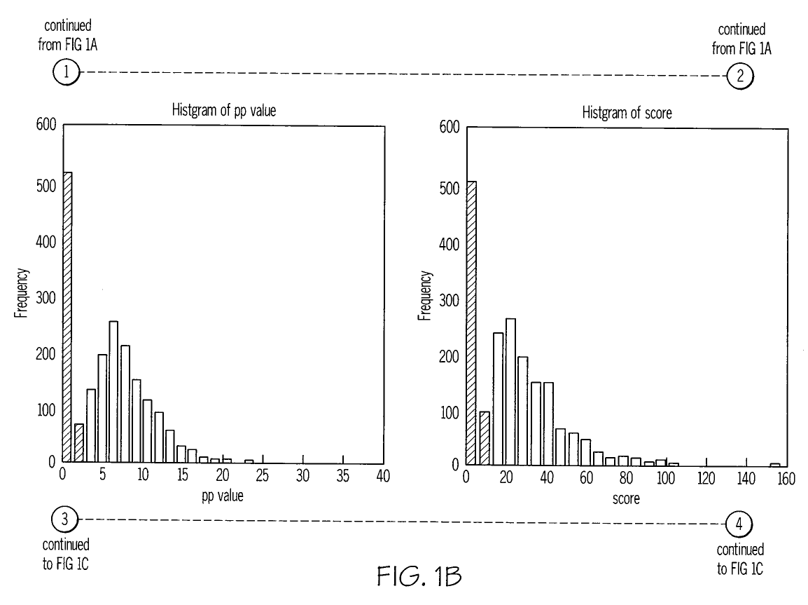

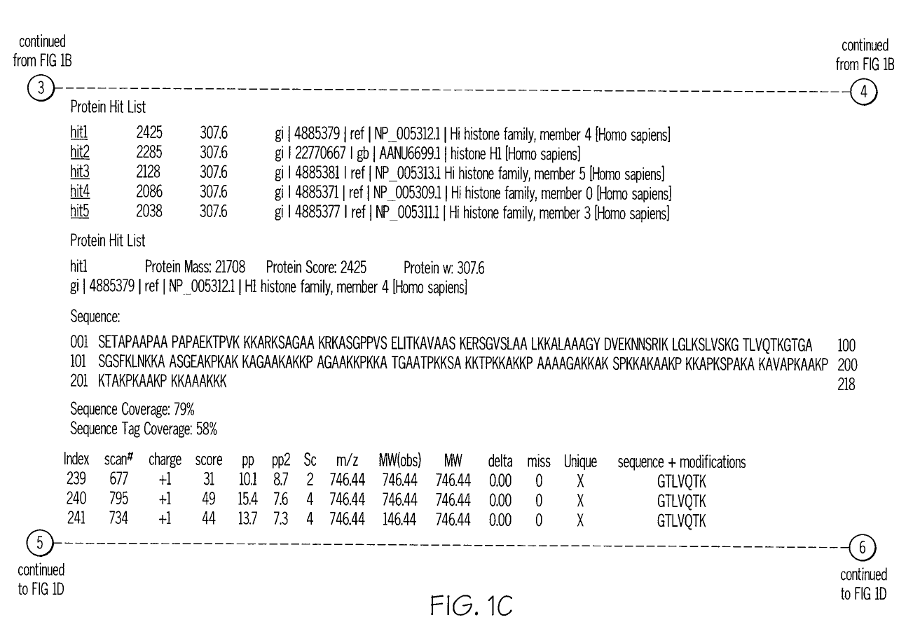

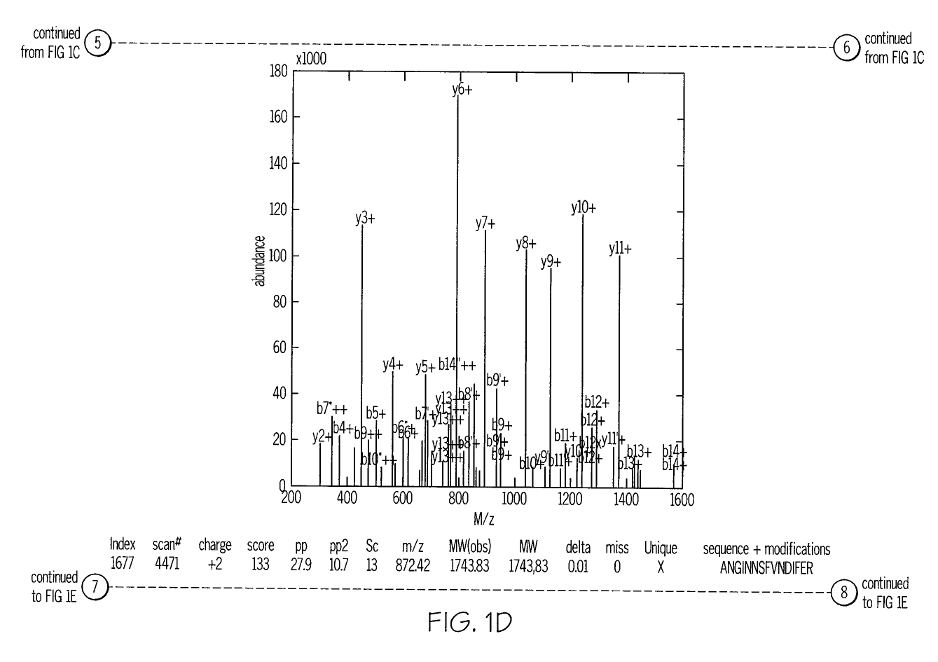

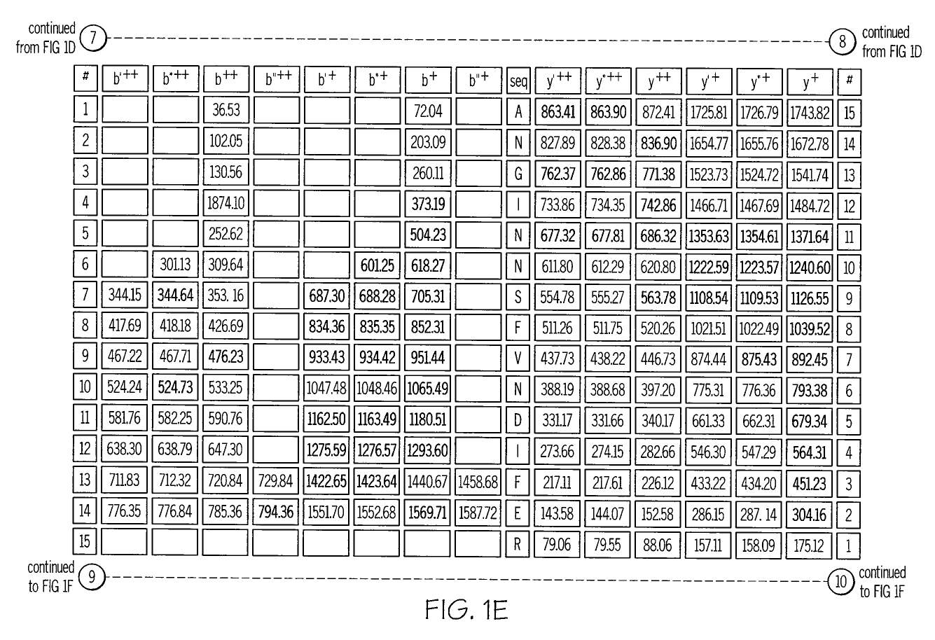

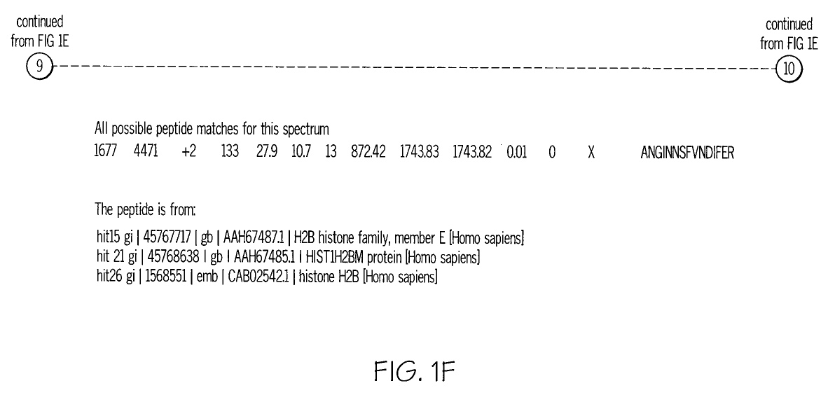

An example of the MS/MS database search program output is illustrated in FIG. 1. For the LTQ and Orbitrap data sets, 1081 out of 2478 and 97 out of 1837 spectra were identified as high quality peptide matches.

The MS/MS database search program employs a mathematically rigorously derived statistical model that statistically calculates the possibility that the match is a random occurrence. Raising mass accuracy will increase scores of true matches and lower those of random matches at the same time and consequently help discriminate true matches from random matches. Automated database searching is possible and no follow-up manual verification is needed. This feature is comes in handy when searching large data sets where manual verification is not practical.

Multiple Scoring Algorithms

The peptide matching algorithm contains two independent scoring models, including a descriptive model and a statistical model. These models are used to calculate three distinct scores for a peptide match. Each of the scores may be independently used to ascertain the quality of the match. Because each score is distinct, the combination of scores is useful for validating each peptide match. The two models and the application to calculating peptide match scores are described in detail in the following.

### Empirical Scores for Peptide Matches and Protein Hits

Score for Peptide Matches

Descriptive scores do not strictly convey any statistical relevance and may be prone to bias due to scoring parameters. However, they have proven to be useful and generally augment probability based scores. For each peptide match, its score, S, is calculated via the following equation:

$$S = 100 \frac{{\sum }_{i = 1}^{{n}_{match}} {I}_{i} {r}_{match}^{2} max ( 0 , \frac{{n}_{match} - 3}{{n}_{match}} )}{\sqrt{{L}_{pep}}} \qquad ( 1 )$$

where Iᵢ is defined as the standardized abundance of the i^{th} product ion in the experimental spectrum (calculated by dividing the abundance of the i^{th} product by the maximum abundance in the spectrum),

$${\sum }_{i = 1}^{{n}_{match}} {I}_{i}$$

is the total standardized abundance of matched product ions, n_{match} is the number of matched product ions, r_{match} is the ratio of standardized abundance of matched product ions to total standardized abundance of the experimental spectrum, and Lₚₑₚ is the length of the peptide in the number of amino acids. Each of these factors contributes to the overall score as follows:

$${\sum }_{i = 1}^{{n}_{match}} {I}_{i}$$

evaluates the quality of the match, r_{match}² introduces a penalty for unmatched product ions,

$$max ( 0 , \frac{{n}_{match} - 3}{{n}_{match}} )$$

is an arbitrary penalty for matches with poor fragmentation, \sqrt{Lₚₑₚ} is an additional penalty for peptides with long sequences and the constant 100 is used arbitrarily to scale scores. By default, scores for a spectrum with less than three matched product ions will be 0 due to the arbitrary penalty. However, the minimum number of matched ions may be changed to any value. Reducing this number is especially valuable for the analysis of singly charged peptides that have characteristic C-terminal aspartic acid fragmentation. The penalty for peptide length is included to normalize the scores. Peptides with longer sequences have more fragment ions and higher empirical scores than shorter sequences. The penalty results in long and short sequences both have similar scores for matches of similar quality. The choices of incorporating squared and square root for the terms n_{match} and Lₚₑₚ were empirically determined from the evaluation of tandem MS data sets collected from LCQ and LTQ-Orbitrap mass spectrometers.
Score for Protein Hits

For true matches, it is assumed the scores follow a normal distribution with a mean of 20 and a variance of 25, i.e. N(20,25). This arbitrary distribution estimates the distributions observed from analysis of several datasets. The expected contribution of each match to the protein score will be

$$S \times {\int }_{S}^{+ ∞} \frac{{ⅇ}^{- {( x - 20 )}^{2} / 50}}{5 \sqrt{2 π}} ⅆ x$$

Thus, the protein score from the descriptively scored matches is calculated from equation 2.

$$protein score = {\sum }_{i} {S}_{i} \times {\int }_{S}^{+ ∞} \frac{{ⅇ}^{- {( x - 20 )}^{2} / 50}}{5 \sqrt{2 π}} ⅆ x . \qquad ( 2 )$$

Probability Based Peptide Scoring Model

In addition to the empirical score, a mass accuracy sensitive probability based scoring model was derived to evaluate peptide matches. The model determines the likelihood that an experimental spectrum match to a theoretical spectrum is a random occurrence. Consider a pair of spectra: one experimental and one theoretical. Wₑ and Wₜ denote their parent masses respectively. In addition, the experimental data contains information regarding the abundance of product ions Iᵢ for each precursor, Wₑ. The model ultimately tests the following two hypotheses: the null hypothesis, H₀, states that a match is random, i.e. the theoretical spectrum is independent of the experimental; and the alternative hypothesis, H_{A}, states that the match is not random, i.e. the theoretical spectrum is related to the experimental one.

Scoring the match is performed in two stages: 1) match Wₑ against Wₜ within the specified precursor ion mass accuracy and 2) match all product ions in the experimental spectrum against the theoretical within the specified mass accuracy. Both stages rely on calculating the probability that the occurrence of an ion within a fixed mass window could be a random occurrence

$$( p = \frac{mass window}{mass range} ) .$$

To match the experimental precursor with that of theoretical peptide we first define the variable q:

$$q = \{ \begin{matrix}1 & {W}_{e} and {W}_{t} match \\ 0 & otherwise\end{matrix} . \qquad ( 3 )$$

Under H₀, the possibility that any precursor ion match (q=1) could be random is given in equation.

$${p}_{1} = \frac{2 {τ}_{pep}}{Π} \qquad ( 4 )$$

In the above equation, τₚₑₚ is the mass accuracy of the precursor ion and Π is the detection range for the precursor ion. For each precursor ion the mass window is defined as ±τₚₑₚ(2×τₚₑₚ). Thus q has a bernoulli (p₁) distribution under H₀. If the precursor ion masses of the pair of spectra do not match (q=0) then the second stage is skipped. If q=1 we proceed to stage 2 where we test the match of the experimental product ion spectrum against the theoretical spectrum.

The variable bᵢ is defined for each product ion, i, in the experimental spectrum as follows:

$${b}_{i} = \{ \begin{matrix}1 & peak i is matched \\ 0 & otherwise\end{matrix} . \qquad ( 5 )$$

Under H₀, all matched Product ions are random occurrences. The probability that a product ion randomly matches any of the product ions in the theoretical spectrum is:

$${p}_{2} = \frac{{Π}_{theo}}{Π} \qquad ( 6 )$$

where Πₜₕₑₒ is the total coverage of the detection range for all product ions in the theoretical spectrum and Π is the MS/MS detection range. It is assumed that Π is the same as the precursor ion mass range. However, for instruments that have a dynamic detection range assuming a fixed value Π will result in more conservative scores. For each product ion in the theoretical spectrum, the mass window is ±τₘₛₘₛ(2×τₘₛₘₛ). If we assume there is no overlap in the product ion mass windows, then Πₜₕₑₒ is calculated using the following equation
Πₜₕₑₒ=2m×τₘₛₘₛ. (7)

The probability that any single matched product ion (bᵢ=1) could be random can be calculated using the eqn. 8

$${p}_{2} = \frac{2 m \times {τ}_{msms}}{Π} \qquad ( 8 )$$

where τₘₛₘₛ is the product ion mass accuracy and m is the number of product ions within the detection range in the theoretical spectrum. Because the theoretical spectrum is independent of the experimental under H₀, all bᵢ (i=1, 2 . . . , n) are assumed to have an identical and independent bernoulli (P2) distribution under H₀. The model is then used to perform two distinct tests. Each uses a different approach to evaluate the quality of a match: number of matched product ions x and total abundance of matched product ions Y.
The pp Score

The model is used to evaluate whether the number of matched product ions in an experimental spectrum could be a random occurrence. For all spectra whose precursor ion masses match, i.e. q=1, the variable x is defined as the number of product ions in the experimental spectrum that match the theoretical spectrum (eqn. 9) where bᵢ (i=1, 2 . . . , n) is defined in eqn. 5 and n is the number of product ions in the experimental spectrum.

$$x = {\sum }_{i = 1}^{n} {b}_{i} \qquad ( 9 )$$

and variable Y be the total abundance of peaks in the experimental spectrum matched with the theoretical spectrum:

Under H₀, all bᵢ have an identical and independent bernoulli (p₂) distribution. Therefore, x will have a binomial (m, p₂) distribution. Consequently the probability mass function for x is:

$$p ( x ) = \frac{n !}{x ! ( n - x ) !} {{p}_{2}^{x} ( 1 - {p}_{2} )}^{n - x} \qquad ( 10 )$$

where p₂ is calculated from eqn. 6. The p-value, α, is defined as the probability that the quality of a random match between a pair of spectra is greater than or equal to a match observed under H₀.

The pp value, β, is defined as the negative common logarithm of the p-value:
β=−log(α) (11)

We use x to evaluate the quality of a match, such that the p-value is the probability that x for a random match between the pair of theoretical and experimental spectra is greater than or equal to that of the actual match, x=n_{match}, under H₀. The p-value is:

$$α = {\sum }_{x = {n}_{match}}^{n} p ( x ) = {\sum }_{x = {n}_{match}}^{n} \frac{n !}{x ! ( n - x ) !} {{p}_{2}^{x} ( 1 - {p}_{2} )}^{n - x} \qquad ( 12 )$$

the pp value is

$$β = - log ( α ) = - log ( {\sum }_{x = {n}_{match}}^{n} \frac{n !}{x ! ( n - x ) !} {{p}_{2}^{x} ( 1 - {p}_{2} )}^{n - x} ) \qquad ( 13 )$$

The pp2 Score

The second approach evaluates whether the total abundance of matched product ions in the experimental spectrum could be a random occurrence. Y is defined as the total abundance of experimental product ions that match product ions in a given theoretical spectrum:

$$Y = {\sum }_{i = 1}^{n} {I}_{i} {b}_{i} \qquad ( 14 )$$

where Iᵢ is the standardized abundance of the i^{th} product ion in the experimental spectrum and bᵢ is defined in eqn. 5. For clarity we define yᵢ=Iᵢbᵢ to give eqn. 15.

$$Y = {\sum }_{i = 1}^{n} {y}_{i} \qquad ( 15 )$$

However, to complete the test we must know the inherent distribution of Y. This distribution is unknown and thus pp2 values can not be precisely calculated as were the pp values based on the total number of matched product ions. In order to estimate the pp2 value, three assumptions are needed:

1. Iᵢ is identically and independently distributed across product ions in the experimental spectrum,

2. bᵢ is uncorrelated with Iᵢ in the experimental spectrum,

3. the number of product ions, n, in the experimental spectrum is large (n>30).

Under assumption 1, the mean μ_{I} and variance σ_{I}² for the distribution of Iᵢ are estimated by:

$${\hat{μ}}_{I} = \frac{1}{n} {\sum }_{i = 1}^{n} {I}_{i} \\ {\hat{σ}}_{I}^{2} = \frac{1}{n - 1} {\sum }_{i = 1}^{n} {( {I}_{i} - {\hat{μ}}_{I} )}^{2} \qquad ( 16 )$$

Since yᵢ=Iᵢbᵢ, according to assumption 2 yield equation 17 under H₀,

$$\begin{matrix}{μ}_{y} = {E}_{y} ( {y}_{i} ) = {E}_{I} ( {E}_{y \vert I} ( {y}_{i} \vert {I}_{i} ) = {E}_{I} ( {p}_{2} {I}_{i} ) = {p}_{2} {μ}_{I} \\ {σ}_{y}^{2} = {E}_{y} ( {y}_{i}^{2} ) - {{E}_{y} ( {y}_{i} )}^{2} = {E}_{I} ( {E}_{y \vert I} ( {y}_{i}^{2} \vert {I}_{i} ) ) - {( {p}_{2} {μ}_{I} )}^{2} \\ = {E}_{I} ( {μ}_{{y}_{i} \vert {I}_{i}}^{2} + {σ}_{{y}_{i} \vert {I}_{i}}^{2} ) - {p}_{2}^{2} {μ}_{I}^{2} \\ = {E}_{I} \{ {( {I}_{i} {p}_{2} )}^{2} + {I}_{i}^{2} {p}_{2} ( 1 - {p}_{2} ) \} - {p}_{2}^{2} {μ}_{I}^{2} = {E}_{I} ( {p}_{2} {I}_{i}^{2} ) - {p}_{2}^{2} {μ}_{I}^{2} \\ = {p}_{2} ( {μ}_{I}^{2} + {σ}_{I}^{2} ) - {p}_{2}^{2} {μ}_{I}^{2} = {p}_{2} ( 1 - {p}_{2} ) {μ}_{I}^{2} + {p}_{2} {σ}_{I}^{2}\end{matrix} \qquad ( 17 )$$

Thus, μ_{y} and σ_{y}² can be estimated as
\hat{μ}_{y}=p₂\hat{μ}₁ (18)
\hat{σ}_{y}²p₂(1−p₂)\hat{μ}_{I}²+p₂\hat{σ}_{I}²

According the central limit theorem, Y is approximately a normal distribution with the following parameters under assumption 3, i.e. when n is large (n>30)
μ_{Y}=nμ_{y} (19)
σ_{Y}²=nσ_{y}²
The resulting probability density function is given in equation 20.

$${f}_{Y} ( Y ) = \frac{{ⅇ}^{- {( Y - {μ}_{Y} )}^{2} / ( 2 {σ}_{Y}^{2} )}}{\sqrt{2 π} {σ}_{Y}} \qquad ( 20 )$$

And μ_{Y} and σ_{Y}² are estimated by equation 21.
\hat{μ}_{Y}=n\hat{μ}_{y}=np₂\hat{μ}_{I} (21)
\hat{σ}_{Y}²=n\hat{σ}_{y}²=n{p₂(1−p₂)\hat{μ}_{I}²+p₂\hat{σ}_{I}²}=np₂(1−p₂)\hat{μ}_{I}²+np₂\hat{σ}_{I}²

The p-value, α, is the probability that Y for a random match is greater than or equal to that of the actual match, I_{match}, under H₀. The p-value becomes:

$$α = {\int }_{{I}_{match}}^{+ ∞} {f}_{Y} ( x ) ⅆ x = {\int }_{{I}_{match}}^{- ∞} \frac{{ⅇ}^{- {( x - {μ}_{Y} )}^{2} / ( 2 {σ}_{Y}^{2} )}}{\sqrt{2 π} {σ}_{Y}} ⅆ x \qquad ( 22 )$$

and it is estimated by

$$α = {\int }_{{I}_{match}}^{+ ∞} \frac{{ⅇ}^{- {( x - {\hat{μ}}_{Y} )}^{2} / ( 2 {\hat{σ}}_{Y}^{2} )}}{\sqrt{2 π} {\hat{σ}}_{Y}} ⅆ x \qquad ( 23 )$$

resulting in the pp2 value, β, as follows:

$$β = - log ( α ) = - log ( {\int }_{{I}_{match}}^{+ ∞} \frac{{ⅇ}^{- {( x - {\hat{μ}}_{Y} )}^{2} / ( 2 {\hat{σ}}_{Y}^{2} )}}{\sqrt{2 π} {\hat{σ}}_{Y}} ⅆ x ) \qquad ( 24 )$$

The pp2 value can be estimated by equation 17 very efficiently. However the real distribution of Y is more tailed to larger values than the normal distribution. Therefore, pp2 values are overestimated when they are large.

The decoy reversed human database creates ˜1000 times as many theoretical peptides as the bovine histone database. False positive peptide matches from the bovine histone database were assumed to be negligible. Therefore, the peptide matches returned from the bovine histone database were considered as true positives (TPs) while those from the decoy database were considered as false positives (FPs).

Abundance Based Scoring via Monte Carlo Simulation

The peptide abundance scoring model based on MCS was derived from the probabilistic model used to calculate pp2 scores in the MassMatrix software as described previously. However, in this implementation the score was calculated via MCS. In brief, two stages were involved in scoring a peptide match in the model: 1) match experimental precursor mass against theoretical precursor mass within the specified precursor ion mass accuracy and 2) match all product ions in the experimental spectrum against the theoretical ions within the specified product ion mass accuracy. For potential peptide matches that pass stage 1, the model then tests the following two hypotheses in stage 2: the null hypothesis, H₀, which states that a match is random, and the alternative hypothesis H_{A}, which states that the match is not random.

Based on the probabilistic model described previously, the variable bᵢ is equal to 1 if i^{th} product ion in the experimental spectrum matches any theoretical product ion and 0 otherwise. Under H₀, all bᵢ (i=1, 2 . . . , n) have an identical and independent bernoulli (p₂) distribution with

$${p}_{2} = \frac{2 m ⨯ {τ}_{msms}}{Π} ,$$

where τₘₛₘₛ is the product ion mass accuracy, m is the number of product ions within the detection range in the theoretical spectrum and n is the number of product ions in the experimental spectrum. The variable

$$x = {\sum }_{i = l}^{n} {b}_{i}$$

is defined as the number of product ions in the experimental spectrum that match the theoretical spectrum. Under H₀, x has a binomial (m, p₂) distribution

$$Y = {\sum }_{i = l}^{n} {I}_{i} {b}_{i} = {\sum }_{j = 1}^{x} {I}_{j}$$

is defined as the total abundance of experimental product ions that match product ions in a given theoretical spectrum.

The model calculates the p value, α, which is the probability that the total abundance of matched product ions in the experimental spectrum for a random match is greater than or equal to that of the actual match, I_{match}, under H₀, i.e.

$$α = P ( Y \geq {I}_{match} ) = {\sum }_{x = 0}^{n} [ P ( Y \geq {I}_{match} \vert x ) p ( x ) ] \qquad ( 25 )$$

where p(x) is the binomial (m, p₂) probability mass function for x and p(x)=Cₙ^{x}p₂^{x}(1−p₂)^{n-x}. The score defined in the model is the negative logarithm of the p value and called pp2_{MCS}, i.e.

$$pp {2}_{MCS} = - log ( α ) = - log \{ {\sum }_{x = 0}^{n} [ P ( Y \geq {I}_{match} \vert x ) {C}_{n}^{x} {{p}_{2}^{x} ( 1 - {p}_{2} )}^{n - x} ] \} . \qquad ( 26 )$$

In order to accurately calculate the score in equation 26, the running time scales exponentially with the input size, n. To overcome this limitation, an algorithm was employed based on MCS to estimate the score which is a linear time algorithm after optimization. The algorithm is summarized as follows:
1. For a given x, randomly pick x product ions from the experimental spectrum without replacement;
2. Calculate the total abundance of the picked x ions and compare it with I_{match};
3. Repeat procedures 1 and 2 for k times;
4. Calculate P(Y≧I_{match}|x) by

$$P ( Y \geq {I}_{match} \vert x ) = \frac{\# of times that the total abundance of picked x ions \geq {I}_{match}}{k} ; \qquad ( 27 )$$
5. Repeat procedures 1, 2, 3, and 4 for all x=0, 1, . . . , n;
6. Calculate the score using equation 26.
   Sequence Tag-Based Scoring Model via Monte Carlo Simulation

Peptide matches may contain consecutive b and/or y ions resulting in sequence tags. In principle, a random peptide match will have randomly matched product ions and should have fewer number of sequence tags than a true peptide match. The sequence tag-based scoring model tests two hypotheses: the null hypothesis, H₀, which states that the match has matched ions randomly distributed across the theoretical ion table, and the alternative hypothesis H_{A}, which states that the match has a nonrandom pattern of matched ions in the theoretical ion table. For a peptide match, N is the total number of theoretical product ions, and M defines the number of theoretical b/y non-neutral loss ions. For the peptide match, n theoretical ions match to the experimental spectrum. The p value, α, of the hypothesis testing is defined as the probability that the number of sequence tags, t, for a random peptide match is greater than or equal to that of the actual match, n_{T}, under H₀, i.e. α=P(t≧n_{T}) . If we define variable z (z=1, 2, . . . , n) as the number of b/y non-neutral loss ions that randomly match to the experimental spectrum given that n theoretical ions match to the experimental spectrum under H₀, we have

$$α = P ( t \geq {n}_{T} ) = {\sum }_{z = 0}^{n} [ P ( t \geq {n}_{T} \vert z ) p ( z ) ] . \qquad ( 28 )$$

where p(z) is the hypergeometric (m, M, N) probability mass function for z, i.e.

$$p ( z ) = \frac{{C}_{M}^{z} {C}_{N - M}^{n - z}}{{C}_{N}^{n}} . \qquad ( 29 )$$

The score defined in the model, called pp_{tag}, is the negative logarithm of the p value, i.e.

$${pp}_{tag} = - log ( α ) = - log \{ {\sum }_{z = 0}^{n} [ P ( t \geq {n}_{T} \vert z ) \frac{{C}_{M}^{z} {C}_{N - M}^{n - z}}{{C}_{N}^{n}} ] \} . \qquad ( 30 )$$

Again this problem cannot be practically solved and a linear time algorithm based on MCS must be applied to estimate the score. The algorithm we developed is summarized as follows:
1. For a given z, randomly pick z product ions from the M b/y non-neutral loss ions in the theoretical ion table without replacement;
2. Calculate the number of b and y sequence tags, t, based on chosen b and y ions and compare it with n_{T};
3. Repeat procedures 1 and 2 for k times.
4. Calculate P(t≧n_{T}|z) by

$$P ( t \geq {n}_{T} \vert z ) = \frac{£ of times that t \geq {n}_{T}}{k} . \qquad ( 31 )$$
5. Repeat procedures 1, 2, 3, and 4 for all z=0, 1, . . . , n
6. Calculate the score using equation 30.
   Estimation Precision and Computational Expense of MCS Scoring Models

The estimation precision of the scores obtained via MCS depends on the sampling size of each simulation step, k. Increasing the sampling size in MCS will improve the precision of the estimation. However it will also raise the computational expense. The optimal sampling sizes for the two models were determined empirically using an actual tandem MS data sets containing 3068 tandem mass spectra collected from an LCQ Deca XP mass spectrometer. 413 tandem mass spectra were scored by the two MCS based scoring models. The scores for each spectrum were recalculated 100 times by the models to determine the relative standard deviation

$$( RSD = \frac{standard deviation}{mean} \times 100 \% ) .$$

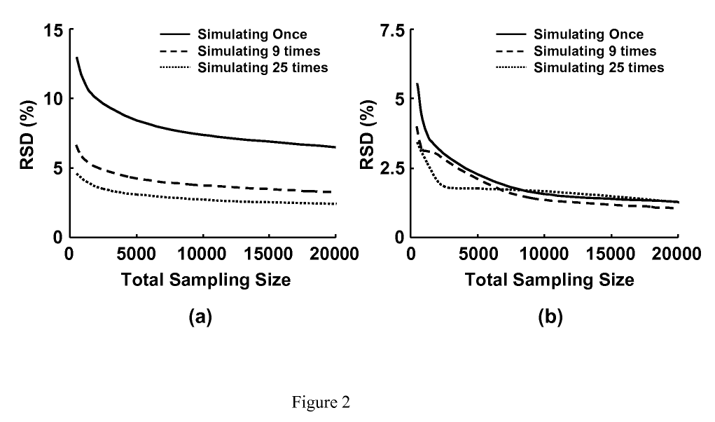

FIG. 2 shows the average RSD of the two scores for the mean 413 spectra. It can seen that for the abundance based score, the precision was poor (RSD >6%) even at very large sampling size. A simple variance reduction technique was then applied in which the simulation was performed R times and the score was estimated by the mean of the estimates from the R simulations. The computational expense depended on the total sampling size of the simulation, which is R multiplied by k. It can be seen from FIG. 2 that the variance reduction technique improved the estimation precision for both models at small and median total sampling sizes. RSD for different R converges as total sampling size approaches infinite in theory. However, RSD of pp_{tag} for R equal to 1, 9 and 25 did not converge completely as shown in FIG. 2b due to the variance of calculating RSD. The actual implementation of the two models in MassMatrix employs the sampling size of k=200 for each step and repeats each simulation for R=25 times for each spectrum, i.e. the total sampling size is equal to 5000. The RSD was improved to 3.1% for the abundance based MCS model and 1.8% for the sequence-tag based MCS model. Therefore, the two MCS models gave scores with reasonable precisions and are of practical use in tandem mass spectrometric database searching.

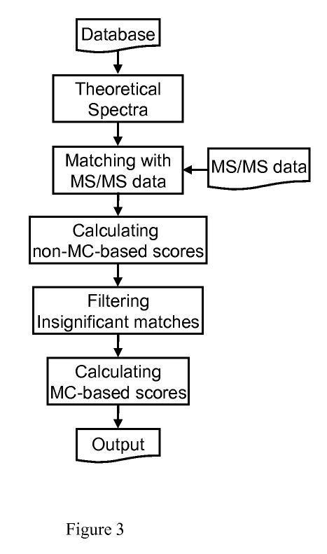

MCS-based algorithms normally suffer high computational expense. For the MCS models implemented in MassMatrix with a total sampling size of 5000, the computational expense for processing 1000 peptide matches was ˜10 sec for abundance based scoring and ˜4 sec for sequence tag based scoring on a single AMD 3200+(2.2 GHz) PC after optimization for speed. Therefore, it was not practical to use MCS-based models to filter and score millions or even billions of potential peptide matches from a real database search. Therefore, potential peptide matches must be pre-filtered by non-MCS-based empirical and statistical models described previously. The non MCS based scoring models take constant time to score a peptide match and are normally hundreds of thousands times faster than MCS-based models. For this reason, MCS-based models were applied to potential peptide matches after pre-filtering the matches by other models (FIG. 3). In this way, database search program need less than 1 minute to calculate scores from MCS-based models in a typical search. For example, the computational expense for processing the testing data set with 3068 spectra was ˜4 sec for the abundance based MCS model and ˜2 sec for the sequence tag-based MCS model. Since all statistical scores from the MCS models and other statistical models in MassMatrix are the negative logarithm of the probability that the peptide match is random, they all share the same standard to determine if a peptide match is significant.

Evaluation of Scoring Models Based on Monte Carlo Simulation

The MCS-based scoring models were evaluated by searching a data set from 23 runs of bovine histone peptide mixture on an LCQ Deca XP mass spectrometer. The results were compared with those from the other statistical models in MassMatrix as described previously. The dataset was searched against a database with a bovine histone database and the reversed human database appended as decoy sequences. The decoy database was much larger than the bovine histone database and creates ˜1000 times as many theoretical peptides as the bovine histone database does. From the combined data set, 13,458 out of 62,117 spectra were scored as potential peptide matches. The summary of the search results is listed in Table 1.

TABLE 1

| | | | | Percentage | Percentage |
|---|---|---|---|---|---|
| | | Spectra | Spectra | of spectra | of spectra |
| charge | Spectra | of TPs | of FPs | of TPs | of FPs |
| +1 | 36196 | 1404 | 2652 | 3.9% | 7.3% |
| +2 and +3 | 25921 | 4042 | 5360 | 15.6% | 20.7% |

Based on the threshold for protein scores automatically set by MassMatrix, there were 53 significant protein hits with protein scores above the threshold and 2144 insignificant protein hits with protein scores below the threshold. Among 53 significant protein hits outputted by the program, the top 51 protein hits were from bovine histone database and the last two were from the decoy reversed human database. Among 2144 insignificant protein hits, 5 were from the bovine histone database and 2139 were from the decoy database. Peptide matches assigned to all protein hits (significant or insignificant) were considered when evaluating the scoring models.

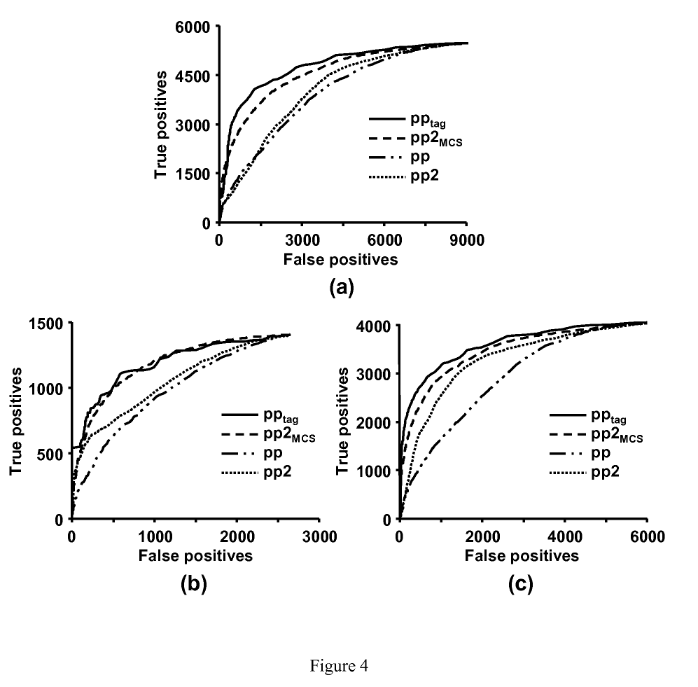

The MCS-based models were evaluated by using receiver operating characteristic (ROC) analysis. Because false positive peptide matches from bovine histone database were considered negligible due to large decoy database, peptides from bovine histone were considered as TPs and those form decoy database were considered as FPs. The results of ROC analysis for the two MCS-based models are shown in FIG. 4a.

The pp2_{MCS} from the abundance-based MCS scoring model was based on the exactly same statistical model as the pp2 score described previously. The only difference between the two scores is that pp2_{MCS} score is calculated via MCS and pp2 score is calculated by use of the central limit theorem. It can be seen from the ROC analysis that the pp2_{MCS} score outperforms the pp2 score. This is because that pp2 value estimated by the central limit theorem cannot evaluate the quality of matches with a small number of product ions and the pp2 scores are normally overestimated when the score is >16 due to assumptions used in the central limit theorem model. The MCS-based models use random sampling methods to estimate the scores without assuming any distribution for any variables involved. Since the distribution of abundance of peaks in tandem mass spectra varies from one spectrum to another, MCS is a better solution for abundance based scoring models in tandem mass spectrometric data analysis.

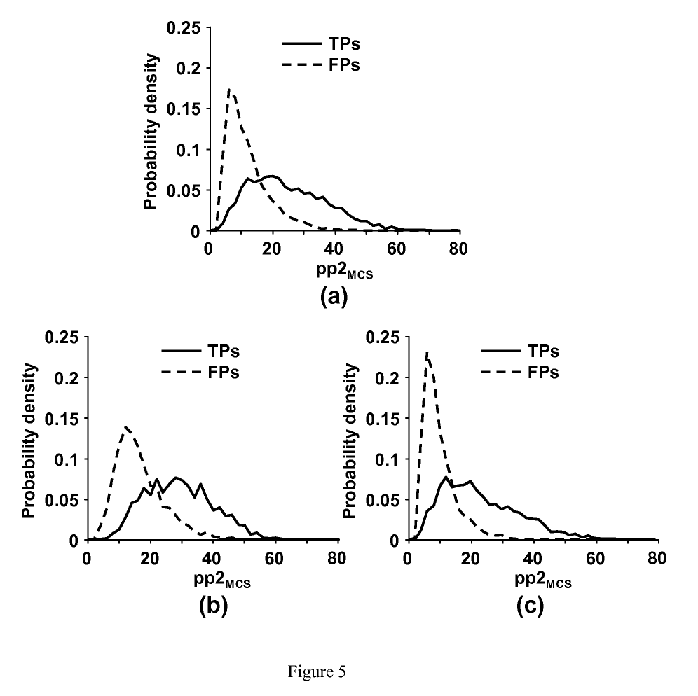

The pp score in MassMatrix is not abundance based and can be solved without assumptions. However, the pp score ignores the information of peak abundance. Interestingly, MCS-based pp2_{MCS} score performed better than the pp score as shown in FIG. 4a. This is exciting in that the pp2_{MCS} does not know anything a priori regarding the expected abundance profile. The distribution of pp2_{MCS} for TPs and FPs is shown in FIG. 5a. At a mass accuracy of 0.8 Da, there was overlap between the distributions of TPs and FPs for pp2_{MCS}. However, it can be seen that TPs tend to have larger pp2_{MCS} scores than FPs.

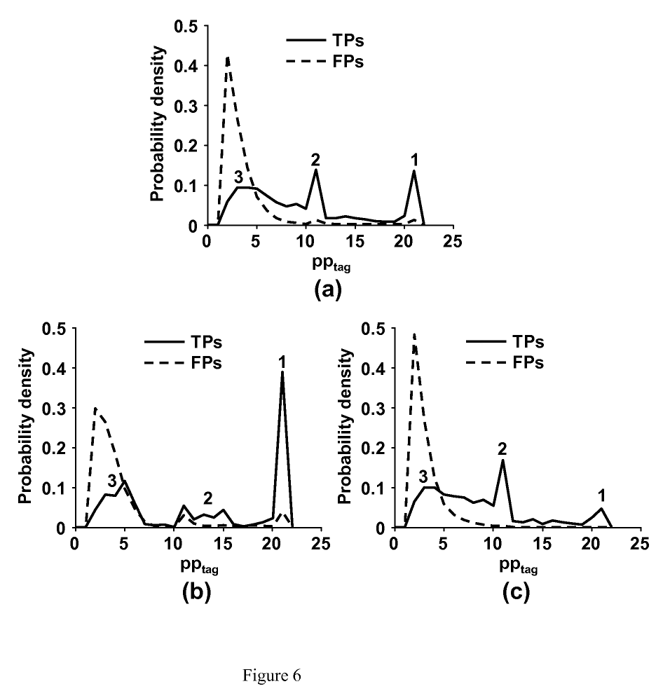

The sequence tag-based model is also a MCS-based statistical model. Moreover, it takes into account the sequence tags inferred from the tandem mass spectra. Surprisingly, it outperformed all other statistical models and served as the best standard to discriminate TPs from FPs even at a low mass accuracy. FIG. 6a shows the distribution of pp_{tag} scores for TPs and FPs. For FPs, pp_{tag} had a much more narrow distribution than pp2_{MCS} score. Furthermore, the overlap between FPs and TPs for pp_{tag} distribution was smaller than that for pp2_{MCS} distribution. This observation was consistent with the ROC analysis and indicated that pp_{tag} performed better than pp2_{MCS} at an accuracy of 0.8 Da. For TPs, the distribution of pp_{tag} was split into three groups labeled by “1”, “2” and “3” in FIG. 6. Peptide matches in groups 1 and 2 had very high scores and narrow distributions. Those peptides had a good ladder of b/y ions that matched the experimental spectra and were well separated from false positive matches. Peptide matches in group 3 normally had a moderate to poor ladder of b/y ions and overlapped with false positive matches.

Effect of Charge on Models Based on Monte Carlo Simulation

It is shown in Table 1 that 15.6% of doubly and triply charged spectra were identified as TPs. However, only 3.9% of singly charged ones were identified as TPs. The ratio of FPs to TPs for singly charged spectra was also higher than that for doubly and triply charged ones. ROC curves for spectra with different charges showed that all statistical scoring models in MassMatrix performed better for doubly and triply charged spectra than singly charged ones (FIGS. 4b & 4c). Therefore, singly charged spectra were less favorable due to their poorer fragmentation when compared with doubly and triply charged ions. The charge state of tandem mass spectra also affected their scores from the MCS-based scoring models. For the abundance based MCS model, the pp2_{MCS} score distributions for doubly and triply charged matches were narrower than those for singly charged ones and the score distribution of TPs was better separated from that of FPs for doubly and triply charged matches than for singly charged ones. This is consistent with the ROC analysis and indicated that the model performed better for doubly and triply charged spectra.

For the sequence tag-based MCS scoring model, the score distributions for singly charged matches and doubly and triply charged ions were quite different. For TPs, the probability density of the group 1 for singly charged matches (FIG. 6b) was much higher than that for doubly and triply charged ones (FIG. 6c). Singly charged peptides had only one set of b/y ions with +1 charge. Therefore, high quality matches had almost a complete set of b/y ions matching to experimental data. Doubly and triply charged peptides had at least two sets of b/y ions with +1 and +2 charges. Doubly and triply charged matches with high quality normally had only one set of b/y ions out of more than two sets of b/y ions in the theoretical product ion table matching to experimental data and had pp_{tag} score lying in group 2 instead of group 1. Since both group 1 and group 2 matches were well separated from the FPs, the charge effect on the model did not greatly affect the performance of the model. Due to the fact that singly charged FPs had a broader distribution than doubly and triply charged FPs, group 3 for singly charged TPs almost completely overlapped with the distribution of FPs, and there was also some overlap between groups 1 and 2 for singly charged TPs and FPs (FIG. 6a). This effect lead to the poorer performance of the model for the singly charged peptides than for doubly and triply charged ones. In general, the score distributions for TPs and FPs were better separated for doubly and triply charged matches than for singly charged ones. Therefore, the model performed much better for doubly and triply charged spectra than singly charged, which confirms the ROC analysis results.

Two new statistical scoring models based on MCS have been developed to evaluate peptide matches in tandem mass spectrometric data and incorporated in a database search program, MassMatrix. The first model is abundance based and evaluates the peptide matches based on the total abundance of the matched peaks in the experimental spectra. It outperforms the non-abundance based scoring model in MassMatrix. The model also outperforms the abundance based model that employs the central limit theorem. This performance gain is because the MCS-based model does not involve any simplification and makes no additional assumptions for the statistical model in order to calculate the statistical scores as other methods do. The second MCS-based model accounts for sequence tags inferred from the tandem mass spectra. This model outperforms all the other statistical models and serves as the best standard in MassMatrix for tandem mass spectrometric data with a low mass accuracy of ˜0.8 Da. Since the two models are based on different criteria, they are complimentary to each other and high confidence of peptide identification can be given with significant scores from both MCS-based models.

Models based on MCS are normally of high computational expense. Therefore, peptide matches were pre-filtered by other statistical models prior to score calculation. By applying a simple variance reduction technique, the MCS-based models achieved good estimation precisions with RSDs of 3.1% and 1.8% for the abundance-based model and sequence tag-based model respectively. ROC analysis indicates that MCS-based scoring models improved the performance of MassMatrix for the data sets with a low mass accuracy of 0.8 Da.

Distribution of pp Value for Random Matches

When q=0, the algorithm always assigns pp value, β=0 because the experimental and theoretical precursor ions do not match. The cumulative distribution function for pp value when q=0 is shown in equation 32.

$${F}_{β \vert q = 0} ( β ) = \{ \begin{matrix}1 & β = 0 \\ 0 & β > 0\end{matrix} \qquad ( 32 )$$

In statistical hypothesis testing, a p-value for a null hypotheses H₀ is always a uniform distribution on the interval [0,1]. Therefore, the cumulative distribution function for p-value of a random match is continuously distributed as
F_{α|q=1}(α)=α (0≦α≦1). (33)
when q=1. According to the definition of pp value (equation 11), the cumulative distribution function for pp value when q=1 is
F_{β|q=1}(β)=1−10^{-β} (β≧0) (34)
and the probability density function is

$$\begin{matrix}{f}_{β \vert q = 1} ( β ) = \frac{ⅆ}{ⅆ β} {F}_{β \vert q = 1} ( β ) = \frac{ⅆ}{ⅆ β} ( 1 - {10}^{- β} ) \\ = ln ( 10 ) {10}^{- β} ( β \geq 0 ) .\end{matrix} \qquad ( 35 )$$

Matches with pp or pp2 values under a critical value β_{c}>0 are discarded, i.e. their pp values are assigned 0. Thus the distribution of pp value for random matches returned by the algorithm is

$$\begin{matrix}\begin{matrix}\begin{matrix}{F}_{β \vert q = 1}^{′} ( 0 ) = {\int }_{0}^{{β}_{c}} {f}_{β \vert q = 1} ( x ) ⅆ x \\ = {\int }_{0}^{{β}_{c}} ln ( 10 ) {10}^{- x} ⅆ x = 1 - {10}^{- {β}_{c}}\end{matrix} \\ For β \geq {β}_{c} ,\end{matrix} & ( 36 ) \\ {F}_{β \vert q = 1}^{′} ( β ) = {F}_{β \vert q = 1} ( β ) = 1 - {10}^{- β} & ( 37 ) \\ Thus when q = 1 & \\ {F}_{β \vert q = 1}^{′} ( β ) = \{ \begin{matrix}1 - {10}^{- {β}_{c}} & β = 0 \\ 1 - {10}^{- β} & β \geq {β}_{c} .\end{matrix} & ( 38 )\end{matrix}$$

Likewise, it can be specified the unconditional distribution of pp values for random matches as follows. Since q has a bernoulli (p₁) distribution,

$$P ( q ) = \{ \begin{matrix}1 - {p}_{1} & q = 0 \\ {p}_{1} & q = 1 ,\end{matrix} \qquad ( 39 )$$

For β=0, the cumulative distribution function becomes,

$$\begin{matrix}\begin{matrix}{F}_{β} ( 0 ) = {F}_{β \vert q = 0} ( 0 ) \times {P}_{q} ( 0 ) + {F}_{β \vert q = 1}^{′} ( 0 ) \times {P}_{q} ( 1 ) \\ = 1 \times ( 1 - {p}_{1} ) + ( 1 - {10}^{- {β}_{c}} ) \times {p}_{1} = 1 - {p}_{1} {10}^{- {β}_{c}}\end{matrix} & ( 40 ) \\ and for β \geq {β}_{c} > 0 , it becomes , & \\ \begin{matrix}{F}_{β} ( β ) = {F}_{β \vert q = 0} ( β ) \times {P}_{q} ( 0 ) + {F}_{β \vert q = 1}^{′} ( β ) \times {P}_{q} ( 1 ) \\ = 0 \times ( 1 - {p}_{1} ) + ( 1 - {10}^{- β} ) \times {p}_{1} = 1 - {p}_{1} {10}^{- β}\end{matrix} & ( 41 )\end{matrix}$$

The combined cumulative distribution function is thus,

$${F}_{β} ( β ) = \{ \begin{matrix}1 - {p}_{1} {10}^{- {β}_{c}} & β = 0 \\ 1 - {p}_{1} {10}^{- β} & β \geq {β}_{c}\end{matrix} . \qquad ( 42 )$$

When β≧β_{c}, F_{β}(β) is continuous and the probability density function of pp value for random matches is

$$\begin{matrix}{f}_{β} ( β ) = \frac{ⅆ}{ⅆ β} {F}_{β} ( β ) = \frac{ⅆ}{ⅆ β} ( 1 - {p}_{1} {10}^{- β} ) \\ = {p}_{1} ln ( 10 ) {10}^{- β} ( β \geq {β}_{c} ) .\end{matrix} \qquad ( 43 )$$

Confidence Level for pp and pp2 Values

The confidence level can also be determined for both pp and pp2. Suppose there are r theoretical spectra within the protein sequence database. If we assume that all theoretical spectra are uncorrelated, equation 44 gives φ, the number of random matches that have a pp value greater than or equal to β under H₀ for any given experimental spectrum.

$$\begin{matrix}ϕ = {r}_{no} {\int }_{β}^{+ ∞} {f}_{β} ( x ) ⅆ x = {r}_{no} {\int }_{β}^{+ ∞} {p}_{1} ln ( 10 ) {10}^{- x} ⅆ x \\ = {r}_{no} {p}_{1} {10}^{- β} ,\end{matrix} \qquad ( 44 )$$

The confidence level, ψ, is defined as
ψ=−log(φ)=−log(rₙₒp₁10^{-β})=β−log(rₙₒ)−log(p₁) (45)
where β is either the pp or pp2 value, r is the number of theoretical spectra within the protein sequence database, and p₁ is given in equation 4. Confidence levels calculated from pp value and pp2 value are referred as confidence level and confidence level2 respectively.

The confidence level is the negative common logarithm of the expected number of random matches with a pp value bigger than or equal to the one we observe for the corresponding experimental spectrum. Therefore, if the confidence level is below 0, more than one random match for the spectrum is expected and the corresponding match is highly suspect. From equation 45, the pp value is directly related to the confidence value. The confidence level is dependent upon the size of the database and degrades as the number of peptide created from the database increases.

Protein pp Value

The pp model is also used to calculate pp values for protein matches. Let rₚᵣₒₜₑᵢₙ denote the total number of theoretical spectra created from a protein sequence and n_{spectra} denote the total number of experimental spectra in the data set. The cross match of all experimental spectra with theoretical peptides for the protein sequence generates n_{match}_{_{—}}ₚᵣₒₜₑᵢₙ=rₚᵣₒₜₑᵢₙ×n_{spectra} potential matches. The sum of reported pp values of all matches for the protein is calculated from equation. 46.

$$B = {\sum }_{i}^{{n}_{match\_protein}} {β}_{i} . \qquad ( 46 )$$

The statistical model is used to test the following hypotheses: H₀—All peptide matches for a given protein are random and H_{A}—At least one peptide match for a given protein is not random. We assume that r_{spectra} theoretical spectra created from the protein sequence are uncorrelated to each other and that n_{spectra} experimental spectra from the data set are uncorrelated to each other. Since n_{match}_{_{—}}ₚᵣₒₜₑᵢₙ is normally very large, B is approximately a normal distribution with a mean of μ_{B}=n_{match}_{_{—}}ₚᵣₒₜₑᵢₙ×μ_{β} and a variance of σ_{B}²=n_{match}_{_{—}}ₚᵣₒₜₑᵢₙ×σ_{β}² according to the central limit theorem.

According to the distribution of the pp value for random matches described above, the mean and variances of a random match are given by the following equations:

$$\begin{matrix}\begin{matrix}{μ}_{β} = {\int }_{0}^{+ ∞} x {f}_{β} ( x ) ⅆ x = {\int }_{{β}_{c}}^{+ ∞} x {p}_{1} ln ( 10 ) {10}^{- x} ⅆ x \\ = ( \frac{{p}_{1}}{ln ( 10 )} + {p}_{1} {β}_{c} ) {10}^{- {β}_{c}}\end{matrix} & ( 47 ) \\ and & \\ \begin{matrix}{σ}_{β}^{2} = E ( {β}^{2} ) - {μ}_{β}^{2} = {\int }_{0}^{+ ∞} {x}^{2} {f}_{β} ( x ) ⅆ x - {μ}_{β}^{2} \\ = {\int }_{{β}_{c}}^{+ ∞} {x}^{2} {p}_{1} ln ( 10 ) {10}^{- x} ⅆ x - {μ}_{β}^{2} \\ = ( \frac{2 {p}_{1}}{{ln ( 10 )}^{2}} + \frac{2 {p}_{1} {β}_{c}}{ln ( 10 )} + {p}_{1} {β}_{c}^{2} ) {10}^{- {β}_{c}} - \\ {[ ( \frac{{p}_{1}}{ln ( 10 )} + {p}_{1} {β}_{c} ) {10}^{- {β}_{c}} ]}^{2}\end{matrix} & ( 48 )\end{matrix}$$

where p₁ is given in equation 4 and β_{c} is the pp value threshold. Likewise for the sum of pp values for the protein, B, the mean and variance for the distribution under H₀ are given in equation 49:

$$\begin{matrix}{μ}_{B} = {n}_{match\_protein} ( \frac{{p}_{1}}{ln ( 10 )} + {p}_{1} {β}_{c} ) {10}^{- {β}_{c}} & \\ \begin{matrix}{σ}_{β}^{2} = {n}_{match\_protein} \{ ( \frac{2 {p}_{1}}{{ln ( 10 )}^{2}} + \frac{2 {p}_{1} {β}_{c}}{ln ( 10 )} + {p}_{1} {β}_{c}^{2} ) {10}^{- {β}_{c}} - \\ {[ ( \frac{{p}_{1}}{ln ( 10 )} + {p}_{1} {β}_{c} ) {10}^{- {β}_{c}} ]}^{2} \}\end{matrix} & ( 49 )\end{matrix}$$

The p-value for a protein, αₚᵣₒₜₑᵢₙ, is defined to be the probability that the protein hit can have a sum of pp values from all its peptide matches greater than or equal to B under H₀. Thus αₚᵣₒₜₑᵢₙ is given by

$${α}_{protein} = {\int }_{B}^{+ ∞} {f}_{B} ( x ) ⅆ x = {\int }_{B}^{+ ∞} \frac{{ⅇ}^{- {( x - {μ}_{B} )}^{2} / ( 2 {σ}_{B}^{2} )}}{\sqrt{2 π} {σ}_{B}} ⅆ x \qquad ( 50 )$$

and the protein pp value becomes

$$\begin{matrix}protein pp value = - log ( {α}_{protein} ) \\ = - log ( {\int }_{B}^{+ ∞} \frac{{ⅇ}^{- {( x - {μ}_{B} )}^{2} / ( 2 {σ}_{B}^{2} )}}{\sqrt{2 π} {σ}_{B}} ⅆ x )\end{matrix} \qquad ( 51 )$$

Effect of Various Spectral Characteristics on Scoring

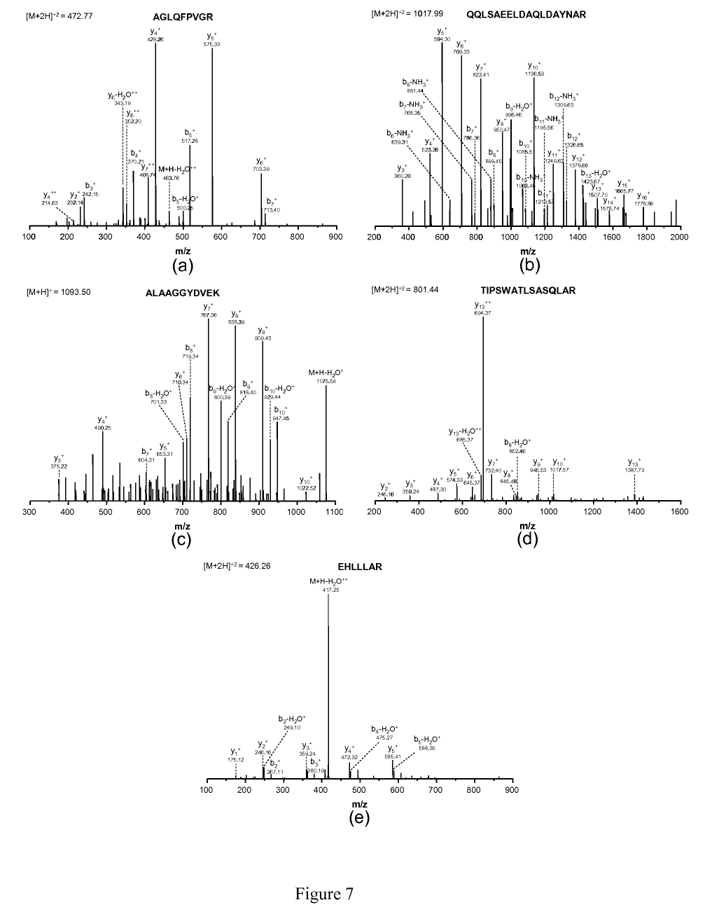

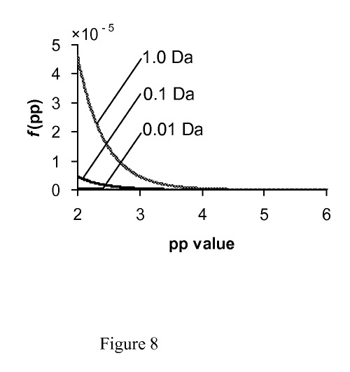

Five example spectra, shown in FIG. 7, are used to illustrate the effect of various spectral characteristics on scoring. All spectra were collected on an LTQ-Orbitrap mass spectrometer (ThermoElectron Finnigan, San Jose, Calif., USA). Precursor and product ions were mass analyzed by the Orbitrap to achieve a mass accuracy of <5.0 ppm. The pp and pp2 values at different mass accuracies (0.01 Da, 0.1 Da and 1.0 Da) were calculated and listed in Table 2.

TABLE 2

| Mass | | | | | | | Confidence | Confidence |
|---|---|---|---|---|---|---|---|---|
| accuracy | Spectrum | p1 | p2 | score | pp | pp2 | level | level 2 |
| 0.01 Da | a | 2.1 × 10⁻⁵ | 7.9 × 10⁻⁴ | 69 | 53.8 | 307.6 | 52.0 | 305.8 |
| | b | 2.1 × 10⁻⁵ | 2.1 × 10⁻³ | 75 | 69.0 | 307.6 | 67.2 | 305.8 |
| | c | 2.1 × 10⁻⁵ | 9.2 × 10⁻⁴ | 59 | 43.0 | 307.6 | 41.2 | 305.8 |
| | d | 2.1 × 10⁻⁵ | 1.5 × 10⁻³ | 19 | 75.2 | 307.6 | 73.4 | 305.8 |
| | e | 2.1 × 10⁻⁵ | 6.5 × 10⁻⁴ | 27 | 36.6 | 307.6 | 34.8 | 305.8 |
| 0.1 Da | a | 2.1 × 10⁻⁴ | 7.8 × 10⁻³ | 77 | 38.6 | 203.9 | 35.8 | 201.1 |
| | b | 2.1 × 10⁻⁴ | 2.1 × 10⁻² | 95 | 46.7 | 157.5 | 43.9 | 154.7 |
| | c | 2.1 × 10⁻⁴ | 9.1 × 10⁻³ | 68 | 26.9 | 274.7 | 24.1 | 271.9 |
| | d | 2.1 × 10⁻⁴ | 1.5 × 10⁻² | 20 | 45.2 | 37.3 | 42.4 | 34.5 |
| | e | 2.1 × 10⁻⁴ | 6.5 × 10⁻³ | 28 | 23.7 | 82.8 | 20.9 | 80.0 |
| 1.0 Da | a | 2.1 × 10⁻³ | 7.8 × 10⁻² | 100 | 22.1 | 21.3 | 18.3 | 17.5 |
| | b | 2.1 × 10⁻³ | 2.1 × 10⁻¹ | 120 | 16.0 | 12.1 | 12.2 | 8.3 |
| | c | 2.1 × 10⁻³ | 9.1 × 10⁻² | 74 | 7.8 | 23.1 | 4.0 | 19.3 |
| | d | 2.1 × 10⁻³ | 1.5 × 10⁻¹ | 55 | 15.5 | 6.1 | 11.7 | 2.3 |
| | e | 2.1 × 10⁻³ | 6.4 × 10⁻² | 36 | 14.7 | 9.0 | 10.9 | 5.2 |

Empirical and statistical scores along with associated parameters (p1 and p2) for each spectrum shown in FIG. 7. The data were obtained for mass accuracies of 1.0 Da, 0.1 Da and 0.01 Da. Confidence levels were calculated based on a search space of 2726345 theoretical peptides. The confidence levels for the pp and pp2 scores are denotes as confidence level and confidence level2.

Mass accuracy tolerances were specified as either relative or absolute for precursor ions but only as absolute tolerances for product ions. Absolute mass accuracy tolerances for product ions are computationally cheaper and yield a reasonable compromise between computational expense and accuracy. Good quality spectra (FIG. 7a, 7b) yielded high empirical and statistical scores as expected. These sequences in FIGS. 7a & 7b illustrate that peptide length has little effect on the scoring. This observation is consistent with the statistical model lack of peptide sequence bias and the peptide length penalty included in the empirical score (eqn. 1). Low quality data (i.e. low signal to noise ratio) can still yield good scores if the most abundant ions are dominated by signal (FIG. 7c). The most challenging spectra are those with few dominant signal peaks. Examples are shown in FIGS. 7d & 7e. These figures show the spectra with one single dominant ion due to N-terminal fragmentation of an internal Pro residue and the neutral loss of H₂O at an N-terminal Glu residue. The empirical scores were poorer for these cases since only a single ion mainly contributes to score (equation 1). However, the pp and pp2 values were not as severely affected and able to accurately discriminate these matches from false positives.

Comparison Between pp and pp2 Values

The pp value is the primary discriminator for quality of matches. The pp2 value can provide a complementary assessment of quality when pp values are suspect. Although the pp and pp2 value have the same statistical basis, there are several differences between them: The pp value is based on the number of matched product ions and the pp2 value is based on the total abundance of matched product ions. The pp value can be underestimated when noise is present in the experimental spectrum especially at low mass accuracy. Because noise normally has lower abundance than product ions, the pp2 value, on the other hand, is generally unaffected. As shown in Table 2, pp value for the spectrum with low signal to noise ratio and majority of noise peaks (FIG. 7c) were affected negatively by the noise peaks and relative low compared with those for normal spectra at mass accuracy of 1.0 Da. However, pp2 value was not affected by the noise peaks.

While pp value can be precisely calculated, there are three assumptions needed to estimate the pp2 values. Assumption 1 and 3 for the pp2 test are not plausible when the number of product ions in the experimental spectrum, n is small. Therefore, pp2 value estimated by the central limit theorem cannot evaluate the quality of matches with a small number of product ions. Furthermore the normal distribution under the central limit theorem is less tailed than the true distribution of Y, pp2 value is normally overestimated when it is large (>16) as shown in Table 2.

From the above discussion, the pp value is more reliable and accurate than the pp2 value under most circumstances, but it can be affected by noise. Under these circumstances, the number of product ions in the spectrum is normally large and pp2 value can be well estimated and complementary to pp value. Thus the combination of the two scores provides an excellent means to ascertain the quality of matches under conditions where one might fail.

Effect of Mass Accuracy on Pp Values

In the pp model, the two most important parameters (p₁, the probability that a theoretical precursor randomly matches the experimental and p₂, the probability that a theoretical product ion randomly matches any product ions in the MS/MS spectrum) are set in accordance with the predetermined mass accuracy of mass spectrometer. These parameters' values decrease as mass accuracy increases. This effect is shown in Table 2. The statistical model specifically takes each parameter into account when calculating the statistical scores. Therefore, these two parameters have a substantial effect on the pp values for both random matches and true matches. Consequently, pp and pp2 values are very sensitive to the accuracy of mass spectrometer.

As is shown in the FIG. 2, the probability of random matches having high pp values is substantially reduced as we increase mass accuracy. Increasing mass accuracy resulted in a shift of the pp value distribution for random matches to lower values. At the same time the pp value distribution for true matches moves to higher pp values. This effect is evident from the pp values in Table 2. As mass accuracy improved, the pp values improved for all peptide matches in FIG. 7. Thus higher mass accuracy improves sensitivity and selectivity for a search and help discriminate true matches from random matches. The probability based algorithm implicitly incorporates mass accuracy into scoring the potential peptide and protein matches. This approach is separate and distinct from algorithms that filter precursor and product ion matches based on mass accuracy. The statistical model involves no empirical parameters and its scores correlate to the probability that a match is a random occurrence. A statistically derived algorithm to rigorously calculate protein scores from the probability based peptide scores was developed. Thus the protein scores reflect the significance of protein matches and can be used to differentiate true protein matches from random matches.

Sample Preparation and Mass Spectrometry

Human histones were isolated from Kasumi-1 cells as described previously. The mixture of core histones was digested using the protease trypsin in 100 mM ammonium bicarbonate buffer. The digested peptides were identified using nano liquid chromatography tandem mass spectrometry (nano-LC-MS/MS). The high mass accuracy data set was obtained from an LTQ-Orbitrap mass spectrometer (ThermoElectron Finnigan, San Jose, Calif., USA) using data-dependent LC-MS/MS. Ions were fragmented by use of collision induced dissociation in the linear ion trap and mass analyzed by the Orbitrap mass analyzer. The high accuracy data set contains precursor and product ions obtained with the Orbitrap.

Database Searching and Searching Parameters

The .RAW data files obtained from the mass spectrometer were converted to .DTA files using extract_msn.exe, a windows console application provided by Thermo Electron (ThermoElectron Finnigan, San Jose, Calif., USA). Tandem MS spectra with more than five product ions were extracted to .DTA files and then merged into .MGF files by use of a perl script. Spectra were not grouped based on precursor mass. The data set was then searched by use of MassMatrix against the NCBInr human databases using the following options: i) Modifications: variable acetylation of lysine, variable acetylation of N-terminus; ii) Enzyme: trypsin; iii) Missed Cleavages: 3; iv) Peptide Length: 4 to 30 amino acid residues; v) Precursor Ion Charge: +1, +2, +3; and VI) A mass tolerance of 0.02 Da and 0.01 Da for the precursor and product ions respectively. The same data set was also evaluated using Mascot and SEQUEST. The search parameters in Mascot and SEQUEST were identical to those in MassMatrix where appropriate. Critical values for scores in the three programs were set as follows: pp or pp2 value >6 in MassMatrix; score >30 in Mascot; and XCorr >1.5 for +1 peptides, >XCorr >2.0 for +2 peptides and XCorr >2.5 for +3 peptides in SEQUEST. If multiple peptide matches for a given spectrum only the highest scoring match was considered. In order to improve the performance of SEQUEST, the protein database was indexed prior to database searches in SEQUEST.

The true positives (TPs) and false positives (FPs) for the three algorithms were determined by searches against a human database containing reversed decoy sequence. The total number of false positive peptide matches was calculated by multiply the number of peptide matches to reversed sequences by two. The number of true positives was then calculated by subtracting total number of false positives from the total number of peptide matches in the forward and reversed databases.

Comparisons with Other Algorithms

In order to test the consensus between MassMatrix and five publicly available search algorithms: Mascot, SEQUEST, Sonar, Spectrum Mill, and X!Tandem, a dataset provided by Kapp et al. was searched using MassMatrix against the Human International Protein Index database (IPI, version 3.19 July 2003, 60397 entries, European Bioinformatics Institute) using the following options: i) Precursor ion tolerance: 3.0 Da; ii) Product ion tolerance: 0.8 Da iii) No fixed or variable modifications; iv) Enzyme: trypsin; v) Missed Cleavages: 2; yl) Peptide Length: 4 to 40 amino acid residues; vii) Precursor Ion Charge: +1; +2, +3; and viii) Maximum Product Ion Charge: +2. The result was compared with those reported by Kapp et al. The searching parameters in MassMatrix were set according to the corresponding parameters that Kapp et al. used for the other five search program. Mascot, SEQUEST, X!Tandem and Sonar Searches were performed against the Human International Protein Index database with following parameters: trypsin-constrained (two missed cleavages); 3.0 Da precursor ion tolerance and 0.5 Da fragment ion tolerance using monoisotopic masses, and ESIIT selected as instrument setting. Searches in SpectrumMill used the same parameters except that the precursor mass tolerance is 2.5 Da and the fragmentation ion tolerance is 0.7 Da.

Search Engine

The MassMatrix algorithm was developed with ANSI C++. The software is portable and has been compiled successfully on personal computers and high-performance clusters running Microsoft Windows or Linux operating systems, for example. A parallel version, MPI MassMatrix, based on the message passing interface (MPI) has been developed for use on distributed memory clusters. A web interface to a limited public form of the algorithm is also available at http://www.massmatrix.net. MassMatrix supports mzDATA format (.XML) and Mascot generic format (.MGF) as input for tandem MS data. Databases must be formatted as FASTA or converted to the MassMatrix database format (.BAS) for protein database.

MassMatrix contains two independent scoring models, including a mass accuracy sensitive statistical model and a descriptive model. These models are used to calculate three distinct scores for a peptide match. Because each score is distinct, the combination of scores is useful for validating each peptide match. Among these three scores, pp value returned from the statistical model is the primary standard used to assess the quality of peptide matches.

MassMatrix searches post-translationally modifications (PTMs) and chemical modifications of the peptide sequence. There are two types of modifications included: 1) fixed modifications, and 2) variable modifications. Fixed modifications are those that modify all occurrences of certain amino acid residues in the protein sequences. Fixed modifications do not add complexity to the database because the search space does not increase. Variable modifications are those that may or may not modify an occurrence of certain amino acid residues in the protein sequences. Variable modifications add complexity as there are a great number of permutations of variably modified peptides for each sequence. MassMatrix searches all possible permutations of modified peptides for each peptide sequence. For example, a peptide sequence with three lysine residues and two serine residues will create (2+1)³×(1+1)²=108 permutations of unmodified and modified peptides when two variable modifications for lysine and one variable modification for serine are allowed. This paradigm results in high computational expense due to the increased number of theoretically possible modified peptides. The need for such a comprehensive search was born from extensive analysis of histone proteins which possess this level of complexity in their patterns of PTMs. MassMatrix was specifically designed to be able to solve such large problems.

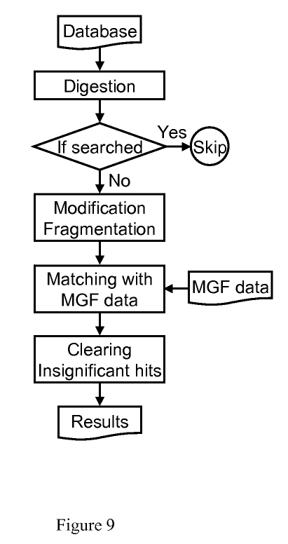

A flow diagram for the database search is shown in FIG. 9. MassMatrix initially digests the protein sequences according to the enzyme or cleavage sites specified by the user. The resulting peptide sequences (and any permutations due to PTMs) are fragmented and then matched against the experimental data. To improve efficiency, it skips redundant peptide sequences. Three scores (score, pp, and pp2 values) are calculated for each potential match using the algorithm described previously. After the scores for all potential matches are calculated, matches below the critical thresholds are discarded. Peptide hits are then matched with its corresponding protein sequences. MassMatrix then outputs the results as html files for portability.

Searches of large data with a large database and many variable modifications are computationally expensive. The number of peptides searched can exceed 10¹⁰ and the job could require days to complete. MPI MassMatrix was designed for use on distributed memory clusters. The search algorithm lends itself to being a parallel application. Since the search processes for each peptide sequence are fully independent, they can be easily split into sub-jobs that are then distributed to many processors on the cluster. The master node initializes the peptide list to be searched and distributes peptide searching as sub-jobs to the slave processors. The master maintains a balanced load among all slaves throughout the job.

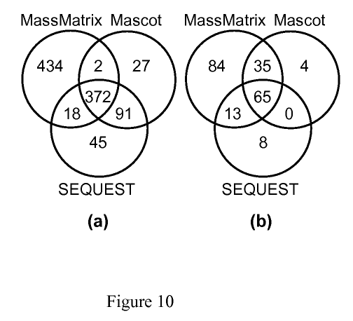

The reliability and consensus of MassMatrix results compared to those of other algorithms was determined by searching the publicly available data set evaluated by Kapp et al. This data set contains 3952 tandem MS spectra obtained from an LCQ Deca XP ion trap mass spectrometer (Thermo-Finnigan, San Jose, Calif., USA). The search parameters were set according to those described previously. Spectra that were not determined to be singly charged were extracted as both doubly and triply charged resulting in 5806 spectra searched. The MassMatrix search was performed at the critical values of pp value=6 and pp2 value=8.826 spectra were identified by MassMatrix with significant peptide matches. The Venn Diagram for peptide matches of the LCQ data set from MassMatrix, Mascot and SEQUEST are shown in FIG. 10(a).

In Kapp's report, 662 out of 3952 spectra were identified with “first pass” matches. Out of these 662 “first pass” matches, the five algorithms: Mascot, SEQUEST, Sonar, Spectrum Mill, X!Tandem, achieved consensus on 349 spectral matches. When factoring in the results from MassMatrix, the consensus peptide lists from all six algorithms drops to 345. The 4 spectra that were different had low signal to noise ratio, and did not appear to be good matches by manual validation.

The consensus results between MassMatrix and each search algorithm were individually compared. MassMatrix achieved high levels of consensus with each algorithm as follows: a) 498 spectra out of all 660 Mascot “first pass” matches; b) 519 out of 662 SEQUEST matches; c) 453 out of 646 Sonar matches; d) 428 out of 544 Spectrum Mill matches; and e) 434 out of 557 X! Tandem matches. For the 660 “first pass” peptide matches from Mascot, 492 of them were verified by Kapp and et al. as correct identifications. Most of these correct ID (488) were captured by MassMatrix. The other 4 were manually checked and they all have very low signal to noise ratio and of low scores.

Comparison of High Accuracy Search Results

The mass accuracy sensitive scoring model in MassMatrix was evaluated against a high mass accuracy data set and the results were compared with those obtained by the commercial search engines Mascot and SEQUEST. Experimental MS/MS data for a tryptic digest of a protein mixture containing histones were obtained on a ThermoElectron Corp LTQ-Orbitrap mass spectrometer. Histones were chosen as they represent a unique challenge to search engines due to the large number of PTMs. A primary goal in developing MassMatrix was to be able to obtain meaningful results from data that contain a large number of PTMs. Precursor and product ions were mass analyzed in the Orbitrap to achieve high mass accuracy (<5 ppm).

FIG. 10(b) displays the Venn Diagrams for peptide matches from MassMatrix, Mascot and SEQUEST. 197 of 1837 spectra were scored as potential peptide matches in MassMatrix. Mascot returned 104 peptide matches where 100 were found by MassMatrix; SEQUEST returned 86 peptide matches of which 78 were found by MassMatrix. MassMatrix returned 42 protein matches for the data set. In comparison, Mascot returned 37 and SEQUEST returned 35. 35 out of 37 Mascot protein matches and 32 out of 35 SEQUEST protein matches were returned by MassMatrix.

Table 3 lists the searching results for the two data sets from the MS/MS database search program, MassMatrix, and the competitor's product, Mascot. The MS/MS database search program has higher sensitivity and matches more peptides than the competitor's product for both the low accuracy and high accuracy data sets as illustrated in the Venn Diagrams depicted in FIG. 10.

TABLE 3

| | |
|---|---|
| LTQ Data (1.0 Da | Orbitrap Data (0.01 Da |
| Mass Accuracy) | Mass Accuracy) |

| | MassMatrix | Mascot | MassMatrix | Mascot |
|---|---|---|---|---|
| Good Hits | 691 | 137 | 193 | 100 |
| Poor Hits | 390 | 89 | 4 | 4 |
| False Rate | 0.36 | 0.39 | 0.02 | 0.04 |

Further comparison between MassMatrix, Mascot and SEQUEST was made by using receiver operating characteristic (ROC) analysis. The number of TPs and FPs in the ROC curves was determined by searches with the presence of the reversed human database in addition to the human database. The ROC analysis of the data set was performed with and without allowing the modifications of acetylation of K and acetylation of N-terminus. Because the majority of correctly identified peptide matches are unmodified, ROC curves of the data set for MassMatrix, Mascot and SEQUEST without allowed modifications were very similar to those with allowed modifications. The result from ROC analysis for the three programs with allowing modifications of acetylation of K and acetylation of N-terminus is discussed in details below (FIG. 11).

Among the peptides with significant scores returned by MassMatrix, the relative number of false positives was as low as 2%. Furthermore all true positives corresponded to a unique peptide/spectrum match in MassMatrix. MassMatrix achieved better sensitivity (2×) than Mascot and SEQUEST for a given specificity when analyzing high mass accuracy data obtained on the Orbitrap. This result is due to the mass accuracy sensitive scoring model that achieves better separation between the distributions of pp values for the true positives and false positives. A more detailed discussion of this effect is present in the next section.

The false rate in Table 3 is defined as the percentage of false positives among all positives. The MS/MS database search program and the competitor's product showed similar selectivity. The false rate for the MS/MS database search program for the high accuracy data was 2%. The low rate is a result of the scoring algorithm that explicitly includes mass accuracy. Thus the distribution of pp scores for true matches was well separated from that of the pp scores for random matches.

Influence of High Accuracy on Searching Results of the MS/MS Database Search Program

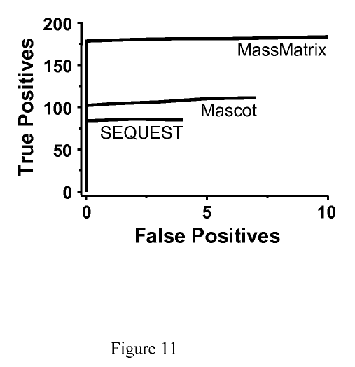

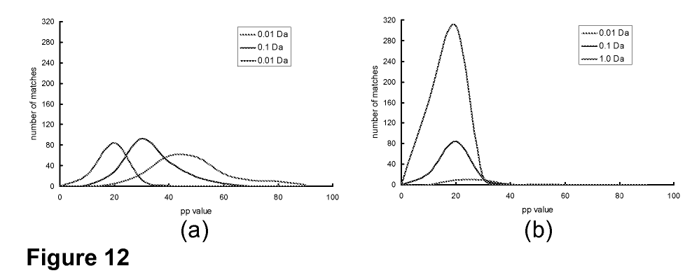

FIG. 12a illustrates the distribution of pp scores for true positives at different levels of mass accuracy. The distribution of the pp scores for true matches shifts to the higher score with increasing mass accuracy. The pp scores for true matches increased by 10 units on average when 10 fold increase in mass accuracy. Thus unlike other algorithms, true matches obtain higher confidence as mass accuracy increases. FIG. 12b illustrates the distribution of pp scores for the false positives at different levels of mass accuracy. The number of false positives substantially decreases at high mass accuracy. This effect is because the pp scores for random matches decrease at higher accuracy and thus fewer random matches are reported by the MS/MS database search program as false positives. Thus raising mass accuracy reduces false positives rate efficiently.

The statistical model in MassMatrix does not provide any benefit or penalty for mass error. Rather the model relies on the occurrence of the mass within the give mass tolerance window. Therefore we were able to simulate the effect of mass spectrometer accuracy on the pp values by altering the mass tolerance used to search the high mass accuracy data set. This approach eliminates any possible factors that may arise due to the use of data sets obtained on different instruments. The distributions of pp values for true and false positives at simulated mass accuracy of 1.0 Da, 0.1 Da and 0.01 Da are shown in FIG. 12. Because the pp values implicitly include mass accuracy in the statistical model, their distribution for true matches improves with the mass accuracy and search tolerance. Using the high mass accuracy data set we observed an increase of 10 in the pp values of true positives for every 10 fold increase in mass accuracy. Thus MassMatrix improves the scores for true matches as the mass accuracy increases. Because the pp value is the negative logarithm of the probability that a match is random the overall statistical confidence in peptide assignment was significantly improved. Additionally the number of reported false positives substantially decreased as mass accuracy increased. In contrast to true matches, the pp scores for false positives decreased at higher accuracy and thus fewer false positives exist above the critical threshold and were reported by MassMatrix. Thus raising mass accuracy has an immediate effect of reducing false positives and improving peptide scores for true positives.

Including additional modifications also increases the number of peptides searched and result in a greater likelihood for peptide false positives. The high mass accuracy data set was also searched against the human database with additional modifications of phosphorylation of serine, threonine and tyrosine The resulting number of peptides searched increased from 6.1 million to 665.7 million. The distraction of additional modification imposed no negative effect on the search results of high accuracy data in MassMatrix.

Influence of Noise on the Searching Results.

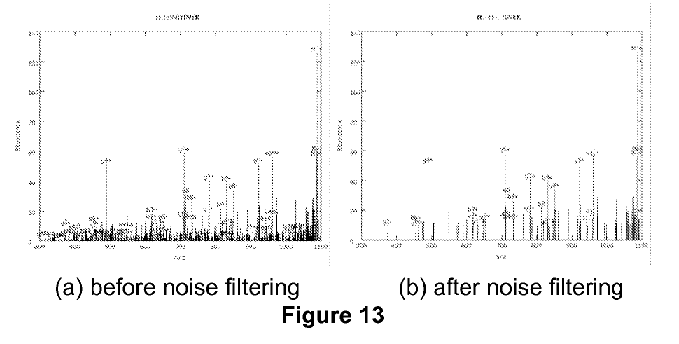

In the MS/MS database search program, pp scores for true matches might be underestimated when spectra are overwhelmed by noise peaks. As is shown in FIG. 13a, for a true match, most of the signal peaks are matched with the theoretical spectrum. However, the majority of the peaks in the spectrum are noise and unmatched. Thus, the pp value is underestimated. In the MS/MS database search program, spectra can be filtered to reduce noise prior to searching. FIG. 13b shows the spectrum match after noise filtering. The majority of noise peaks has been removed by the MS/MS database search program and signal peaks are not affected by the filtering. The filtering improves the quality of the match and raises the pp value from 2.7 to 7.9.

Search of Tandem MS Data Against Decoy Sequences.

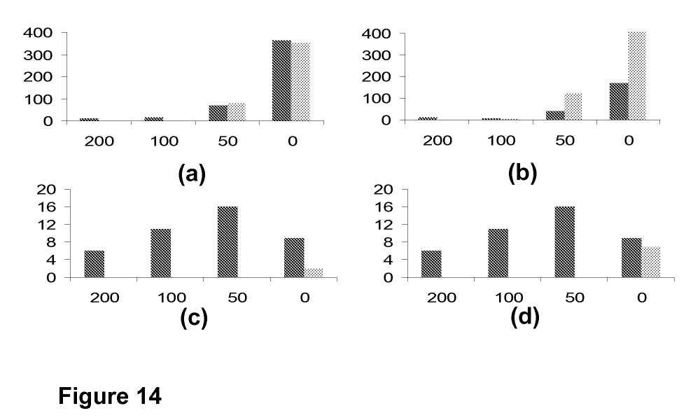

The use of decoy databases appended to the search database results in increase rate of false matches. However, comparison of the top 100 hits from the regular database and the extended database with decoy sequence show that the top hits are virtually identical (Table 4). Low quality spectra are most likely to overlap with score obtained from decoy sequences. The additional decoy sequences have a lesser effect on the identification of proteins. As shown in FIG. 14, nearly all false protein hits from the decoy sequence database have insignificant scores (<100) and all the 28 protein hits with scores >100 were seen in all searches including those using random and reverse decoy sequences. FIG. 14a depicts LTQ data with reversed decoy sequences, FIG. 14b depicts LTQ data with randomized decoy sequences, FIG. 14c depicts Orbitrap data with reversed decoy sequences, and FIG. 14d depicts Orbitrap data with randomized decoy sequences.

TABLE 4

| | Tandem MS Data | | | | | |
|---|---|---|---|---|---|---|
| | LTQ Data | | | Orbitrap Data | | |
| | Decoy sequences present | | | | | |
| | No | Reversed | Randomized | No | Reversed | Randomized |
| Number of peptides | 6.1 | 12.1 | 27.5 | 6.1 | 12.1 | 27.5 |
| searched (million) | | | | | | |
| Peptide matches in | 1080 | 616 | 330 | 197 | 193 | 193 |
| human database | | | | | | |
| Peptide matches | — | 562 | 951 | — | 7 | 34 |
| outside human | | | | | | |
| database | | | | | | |
| Number of top 100 | 100 | 92 | 75 | 100 | 100 | 100 |
| true positives | | | | | | |
| returned | | | | | | |

The additional decoy sequences have nearly no negative effect on the search against Orbitrap data set with at a mass accuracy of 0.01 Da as shown in Table 4 and FIG. 14. All positive identifications from the original search against the human database were present in the search using databases with decoy sequences. Furthermore, a insignificant number of false positives were returned in searches against decoy databases. Thus, at a mass accuracy 0.01 Da, the MS/MS database search program gains relatively high selectivity and can identify peptides with a very low false positive rate even in the presence of decoy sequences. It should also be noted that all searches contained distraction from the inclusion of PTMs. Despite the presence of distraction the false positive rate remained low.

The PC Version of the MS/MS Database Search Program.

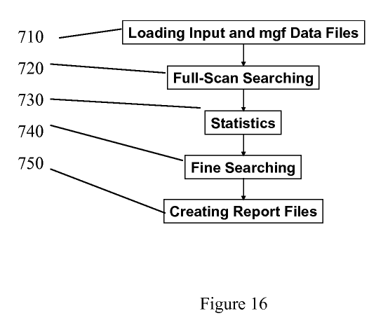

The MS/MS database search program, according to step 710 in FIG. 16, scans the input file and sets up searching parameters: enzyme to use, modifications, database to use, fragmentation type to use and other searching parameters.

According to specified modifications, the MS/MS database search program creates modification operators that will be used in full-scan searching. There are three modification operators used in MassMatrix: MM_PNO, MM_PMOD, and MM_P.

In the MS/MS database search program, 26 letters (A-Z) are used to represent the 26 amino acids (20 common, 4 user defined, N-terminus, and C-terminus) and each of them has a corresponding AA number shown in Table 5.

TABLE 5
AA no for all amino acids and N-terminus, C-terminus

| | Amino Acid | | | | | | | | | | | | |
|---|---|---|---|---|---|---|---|---|---|---|---|---|---|
| | A | C | D | E | F | G | H | I | K | L | M | N | P |
| AA no | 0 | 1 | 2 | 3 | 4 | 5 | 6 | 7 | 8 | 9 | 10 | 11 | 12 |
| | Amino Acid | | | | | | | | | | | | |
| | Q | R | S | T | V | W | Y | J¹ | O¹ | U¹ | X¹ | B² | Z^{J} |
| AA no | 13 | 14 | 15 | 16 | 17 | 18 | 19 | 20 | 21 | 22 | 23 | 24 | 25 |

¹User defined amino acids.
²N-terminus
³C-terminus

MM_PNO: a vector, with 26 elements. Its index is AA no. Each element in the vector is the number of modifications for the corresponding amino acid.
MM_P: a matrix with dimension of 26×max(MM_PNO), where max(MM_PNO) is the maximum number of modifications present on any of the amino acids in the vector
MM_PNO. Each column of MM_P corresponds to all possible masses for the respective amino acid.
MM_PMOD: an integer matrix with dimension of 26×max(MM_PNO). Each column of

MM_PMOD contains the index for each mass in MM_P. So MM_PMOD contain the information relating masses in MM_P to the respective modification. Modification index # for all 48 modifications included in MassMatrix and 3 possible user defined modifications are listed in Table 6.

TABLE 6
Modification no for all modifications included
in the MS/MS database search program*

| | Modification | | | | | |
|---|---|---|---|---|---|---|
| | AceB | BioB | CaAB | PICB | SMAB | TRQB |
| Mod. no | 0 | 1 | 2 | 3 | 4 | 5 |
| | Modification | | | | | |
| | MetB | AceK | BioK | CaAK | PICK | SMAK |
| Mod. no | 6 | 7 | 8 | 9 | 10 | 11 |
| | Modification | | | | | |
| | TRQK | MeK1 | MeK2 | MeK3 | GuaK | UbqK |
| Mod. no | 12 | 13 | 14 | 15 | 16 | 17 |
| | Modification | | | | | |
| | AmiZ | MeEZ | Na + Z | O18Z | CAMC | COMC |
| Mod. no | 18 | 19 | 20 | 21 | 22 | 23 |
| | Modification | | | | | |
| | EthC | MMTC | NIPC | PEOC | ProC | SPEC |
| Mod. no | 24 | 25 | 26 | 27 | 28 | 29 |
| | Modification | | | | | |
| | CATC | MeR1 | MeR2 | OxiM | SulM | PhST |
| Mod. no | 30 | 31 | 32 | 33 | 34 | 35 |
| | Modification | | | | | |
| | PyrE | PyrQ | SulY | TRQY | DANQ | HNEC |
| Mod. no | 36 | 37 | 38 | 39 | 40 | 41 |
| | Modification | | | | | |
| | HSeM | HSLM | MeDE | NaDE | OxHW | PhoY |
| Mod. no | 42 | 43 | 44 | 45 | 46 | 47 |
| | Modification | | | | | |
| | PyrC | UDM1 | UDM2 | UDM3 | — | — |
| Mod. no | 48 | 49 | 50 | 51 | — | — |

A number of modifications is not limited to 51. Additional modifications can be included in the MS/MS database search program if needed.

The MS/MS database search program scans the mgf data file and stores the data set in a special data structure—SPEC_INFO. The data structure can have the structure as follows:

SPEC_INFO data structure:
number of spectra (integer)
scan no for all spectra (vector)
charge of parent ions for all spectra (vector)
mass of parent ions for all spectra (vector)
number of peaks for all spectra (vector)
variances of standardized abundance for all spectra (vector)
m/z data for all spectra (array of vectors)
abundances for all spectra (array of vectors)
standardized abundances for all spectra (array of vectors)

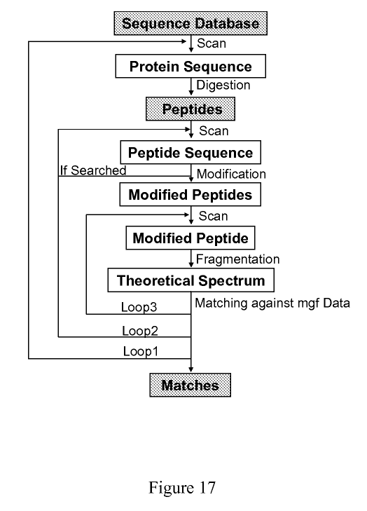

In step 720 of FIG. 16, full scan searching is performed. The overall scheme of the full-scan searching is shown in FIG. 17.

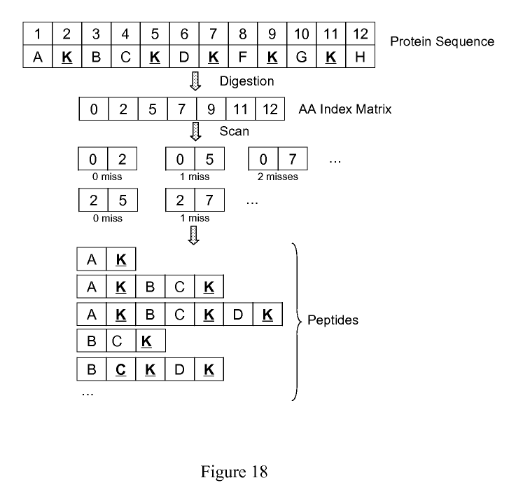

The MS/MS database search program scans the protein sequence database that is specified by the user and picks up one protein sequence at a time. The MS/MS database search program then digests the protein sequence according to the enzyme or cleavage sites specified by the user as illustrated in FIG. 18.

As shown in FIG. 18, according the enzyme used or the cleavage sites specified, the MS/MS database search program scans the protein sequence and stores index numbers of N-terminus, C-terminus and all possible cleavage sites in a matrix. For example, if a protein sequence of “AKBCKDKFKGKH” is digested by an enzyme that cuts the protein after the amino acid K, a matrix of [0, 2, 5, 7, 9, 11, 12] containing N-terminus, C-terminus and all index numbers of amino acid K in the protein sequence will be created and stored in the MSMS database search program. The list of peptides is created by combining any two index numbers in the matrix as the starting and ending points of the peptide respectively. The MS/MS database search program creates one peptides sequence at a time.

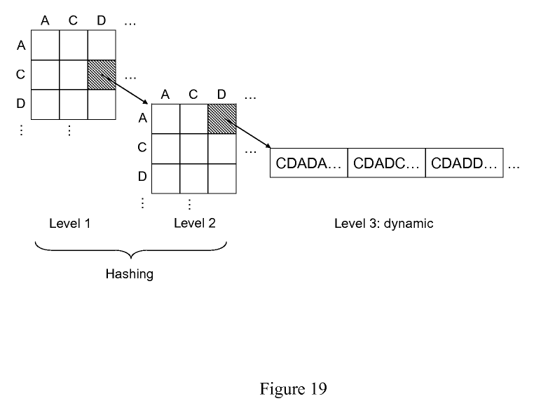

The MS/MS database search program checks if the peptide sequence has been searched before as illustrated in FIG. 19. If true, that peptide will be skipped. Peprecord is a special data structure consists of nested matrices used in the MS/MS database search program to store all the peptides that has been searched. Referring to FIG. 19, Level 1 of peprecord is a two dimensional matrix. Its first dimension is the AA no of the first amino acid of the peptide and second dimension is the AA no of the second amino acid of the peptide (N and C terminus are not included here). Its elements are the index of level 2 of peprecord. Level 2 of peprecord is a two dimensional matrix. Its first dimension is the AA no of the third amino acid of the peptide and second dimension is the AA no of the fourth amino acid of the peptide. Its elements are the index of level 3 of peprecord. Level 3 of peprecord is a one dimensional matrix, i.e. vector, containing a list of sorted peptide sequences. It is dynamic and can be expanded when new peptide sequences come in.

When new peptide sequence is to be searched, the MS/MS database search program checks the sequence against peprecord to see if it has been searched before. If that is the case, the peptide will be skipped and not be searched by the MS/MS database search program again. Otherwise the peptide sequence will be added into peprecord and searched by the MS/MS database search program. A modified hashing and binary searching algorithm is used to accelerate the check/storage process. When a new peptide (target peptide) is to be searched, the MS/MS database search program does a hash search to find its index of level 1 and 2 and then binary searching against the level 3. If the peptide is already in the peprecord, its indexes will be returned, otherwise the indexes of the peptide that is closest to and smaller than the target peptide will be returned and those indexes will be used by the MS/MS database search program to insert the target peptide into the peprecord. Thus, peprecord is maintained as a hashed and sorted list of peptide sequences throughout the searching process. Even if peprecord holds millions of peptide sequences, the MS/MS database search program can finish a checking/storage operation as fast as it does. The initial size of peprecord is about 3 M bytes. After it stores a million of peptide sequences, its size does not exceed 50 M bytes.

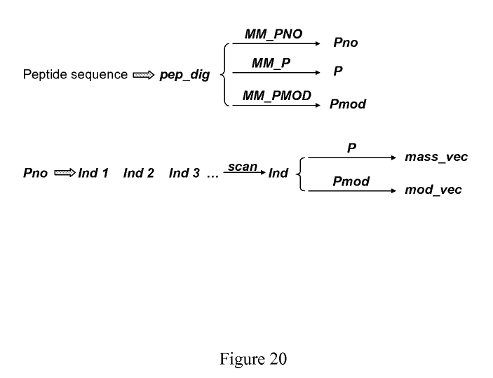

The MS/MS database search program searches with post-translational and chemical modifications to the peptide sequence as illustrated in FIG. 20. The complete list of all modified peptides for the peptide sequence is stored in two special matrices called mass matrix and mod matrix. A small mass matrix containing only around one hundred double precision elements can efficiently store the masses of an unlimited number of modified peptides. Mod matrix is the same size as mass matrix and contains modification information for all modified peptides.

Modification matrices are created by modification operators. The MS/MS database search program converts the peptide sequence into a vector, called pep_dig, containing AA no of amino acids+N-terminus+C-terminus of the peptide. The MS/MS database search program uses the MM_PNO, MM_P and MM_PMOD matrices as operators for pep_dig, to create matrices Pno, P and Pmod, or uses columns in the operators whose column numbers are elements of pep_dig to create target matrices. Pno is the matrix of number of modifications of the amino acids of the peptide. P, called mass matrix, is the matrix of masses of modifications of the amino acids of the peptide. Pmod, called mod matrix, is the matrix of modification index of modifications of the amino acids of the peptide. The Ind matrix is contains indexes of all possible modified peptides for the peptide sequence: Ind has the same dimension as Pno. Each element of Ind can have integer number ranging from 0 to the corresponding element of Pno. All possible combinations create all Ind matrices of modified peptides. Thus there are cumprod (Pno), i.e. cumulative product of Pno, of possible modified peptides for the peptide sequence. The MS/MS database search program creates one Ind matrix at a time. The MS/MS database search program operates P and Pmod on the Ind matrix to create mass_vec and mod_vec for the selected modified peptide. mass_vec and mod_vec are one dimensional matrices, i.e. vectors, that contain masses and modification no of amino acids of the modified peptide respectively.

The MS/MS database search program applies fragmentation to the mass_vec of the selected modified peptide to generate a theoretical spectrum for the peptide. According to the type of fragmentation technique or product ions specified by the user, the MS/MS database search program generates a theoretical spectrum of a, b, c, or x, y, z ions or any combination of those ions for the peptide. Fragmentation comprises cumulative summation of the mass_vec of the modified peptide generates b and y ions. Water and ammonium loss ions are generated via subtracting b and y ions by the mass of water and ammonium. mod_vec is used to determine if there are water or ammonium loss ions for specific b and y ions.

After generating the theoretical spectrum, the MS/MS database search program matches that spectrum against all experimental data stored in the structure SPEC_INFO. The MS/MS database search program matches the theoretical spectrum against all experimental data via two steps. Step 1: the MS/MS database search program searches the theoretical peptide mass against all peptide mass of experimental spectra within the tolerance specified by the user. Step 2: the MS/MS database search program matches the theoretical spectrum against all experimental spectra whose peptide masses are matched in step 1. A modified binary searching algorithm is used here. After that, the MS/MS database search program calculates score, pp and pp2 values for all matches.

In step 730 of FIG. 16, the MS/MS database search program calculates additional scores, confidence level and confidence level 2, and evaluates all matches in spec_matchlist. Insignificant matches with confidence levels below the critical confidence levels are discarded.

If there are multiple matches for a spectrum,

dScore=Score—Scoreₘₐₓ,

dpp=pp−ppₘₐₓ,

dpp2=pp2−pp2ₘₐₓ,

where Scoreₘₐₓ, ppₘₐₓ and pp2ₘₐₓ are the maximum score, pp and pp2 values among all matches for the spectrum. MassMatrix only outputs reasonable matches with dScore, dpp or dpp2 more than or equal to the critical values set by the user.

After the MS/MS database search program statistics, insignificant peptide matches will be discarded. The MS/MS database search program then will check all protein hits. For protein hits with no significant peptide matches will be discarded.

In step 740 of FIG. 16, the MS/MS database search program will recalculate all scores here and outputs all spectra with matched peaks highlighted in PNG format. Sequence tags for all matches are also calculated during fine searching process. In the final result files, sequence tags are highlighted in colors and sequence tag coverage are also reported for each protein hit.

In step 750 of FIG. 16, the MS/MS database search program creates reports in html formats. Scores and pp values for each protein hit are also reported in the result file.

The MS/MS database search program can also be used to search tandem MS data against DNA, RNA or carbohydrate sequence databases other than protein sequence database. In order to do that, users just need to modify amino acids to nucleotides or glucose units.

The MS/MS database search program can also search protein sequences with modifications that can be fragmented or with branches. Two types of fragmental modifications or branches are considered in the MS/MS database search program: 1) Fragmentation of the modification group or the branch occurs only when the sequence bone is unfragmented and 2) Fragmentation of the modification group or the branch occurs when the sequence bone is unfragmented or fragmented.

MPI the MS/MS Database Search Program for Distributed-Memory Clusters.

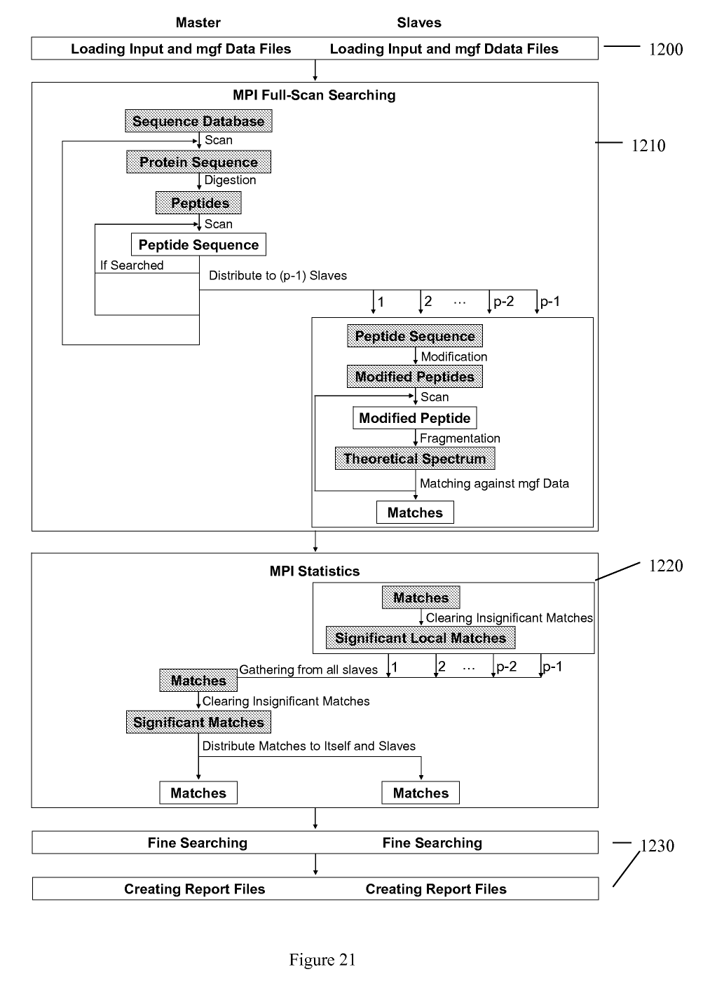

As illustrated in FIG. 21, a faster searching speed is achieved by using a matrix based computational algorithm that is optimized for speed. The speedup on distributed memory clusters is linearly proportional to the number of processes used. This makes it possible to search large data sets against large databases with heavy modifications allowing for identification of PTMs. Low level matrix operations exploit vector processing pipelines. The matrix based algorithm and the matrix library make coding protein digestion, peptide modification and fragmentation models easier and can achieve fast computation speed. This is achieved by using object oriented algorithms. All objects are two dimensional arrays. Digestion, modification and fragmentation processes are implemented by matrix manipulation and calculation functions included in the matrix library. For loops and memory management are hidden in the matrix library. Indirect function call overhead and re-allocation memory are avoided to the greatest extent.

As illustrated in step 1200 of FIG. 21, suppose that MPI_MS/MS database search program uses p processes. One and only one process will be master and the reset of the processes will be slaves. The master and all slaves load the input file, create modification operators and load the mgf data file simultaneously. All processes have same data structures of searching parameters and mgf data and same modification operators.

In full-scan searching, step 1210 of FIG. 21, the master scans the protein sequence database and digests the protein sequences to create peptide sequences. The master scans the peptide sequences and checks if peptide sequences have been searched before. Then the master distributes peptide sequences to slaves that are idle. If no slaves are idle, master waits until at least one slave is idle.

Initially all slaves wait to receive packages from the master. After receiving a peptide sequence from the master, slaves apply modification on the peptide sequence and search all possible modified peptides for the peptide sequence against the mgf data. After finishing the peptide sequence, slaves wait to receive more peptide sequences to search until master tells them to stop. Slaves will keep all searching results in their data structures, spec_matchlist.

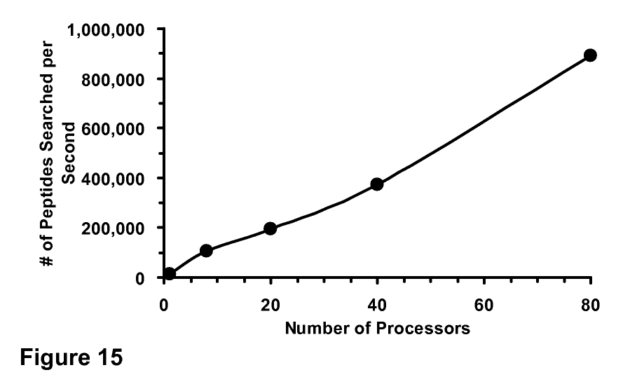

Since scanning protein sequence database and digestion process is much faster than modification and searching process when the MS/MS database search program searches with modifications. It is always the case that the master waits for the slaves to finish their jobs before sending more jobs. So the speedup gained by MPI_MS/MS database search program is almost linearly proportional to the number of processes used as illustrated in FIG. 15.

The search time in MassMatrix is proportional to the number of theoretical peptides searched. Proteins containing redundant sequences in the databases do not increase the search time because redundant peptide sequences are skipped. The searching time also increases with the number of experimental tandem MS spectra. However, this increase is not linear (nearly logarithmic) as all tandem MS spectra are stored in a sorted table and recalled using a binary search algorithm. For the search of Orbitrap data set, MassMatrix and SEQUEST took 61 and 255 sec respectively on a single AMD 3200+ (2.2 GHz) PC. Mascot spent 240 sec on a dual-Intel Xeon server (2.8 GHz×2). Despite the difference in architecture the search results show that MassMatrix performs as well or better than Mascot and SEQUEST with regards to search time. On a modern PC, MassMatrix was able to search >100,000 tandem MS spectra per hour against the human protein database with 2 variable modifications or >100,000 peptides per second against a data set containing 1837 tandem MS spectra.

The scalability of MPI MassMatrix was determined by searching the high mass accuracy data set against a human database with 8 variable modifications. In this example the number of peptides searched exceeded 4×10⁸. The search was performed on a Linux Itanium2 cluster (900 MHz processors). As shown in FIG. 15, the search speed of MPI MassMatrix was nearly proportional to the number of processors used. The speedup showed obvious nonlinearity when more than 40 processors were used. This nonlinear scaling was due to the high communication overhead between the master and slave nodes. The non-ideal scaling was exacerbated by searching a narrow mass tolerance that in turn reduced the number of spectral comparisons on each slave and increased the rate of request between slave and master nodes. Load balancing algorithms are currently being examined to improve the scaling on clusters that exceed 20 processors. The parallel version of MassMatrix achieved a maximum speed of 94,474 tandem MS spectra per hour against the human protein database with 8 PTMs or 5,959,229 peptides per second against a data set containing 1837 tandem MS spectra while running on 80 processors with a peak performance of 72 Gflops.

A new tandem MS database searching program, MassMatrix, was developed that employs a mass accuracy sensitive scoring algorithm. The mass accuracy sensitive scoring algorithm gives rise to higher sensitivity and specificity for searches at high mass accuracy. The high mass accuracy also improves the pp values for true matches, reduces the pp value for random matches and results in improved confidence in peptide identification. The program was first tested and compared with other five search algorithms using a data set that was validated and published by Kapp and et al. It was shown that MassMatrix has consensus with other publicly available programs for searches at low mass accuracy and it returned most of peptide matches from other programs that were validated as correct identification. The peptide matches that MassMatrix missed were then manually checked and found to be of low scores and quality. MassMatrix also returned many unique peptide matches that passed manual validation. The program was also tested and compared with Mascot and SEQUEST using a high mass accuracy data set. The ROC analysis shows that, MassMatrix has a higher sensitivity than Mascot and SEQUEST for the high mass accuracy data. The relative number of false positives for our high accuracy data set was 2% and each spectrum corresponds to a validated unique peptide match. The presence of decoy sequences and additional modifications did not significantly alter the search results. The high confidence achieved when searching high mass accuracy data reduces the requirement for manual validation.

However when no modifications are specified and the MS/MS database search program searches without any modifications, slaves may wait for the master to send their jobs and the speedup gained by the MPI_MS/MS database search program is limited. When searching without modifications, the load of searching jobs is normally small and can be finished within an hour (PC version). So MPI_MS/MS database search program is not recommended when no modifications are specified.

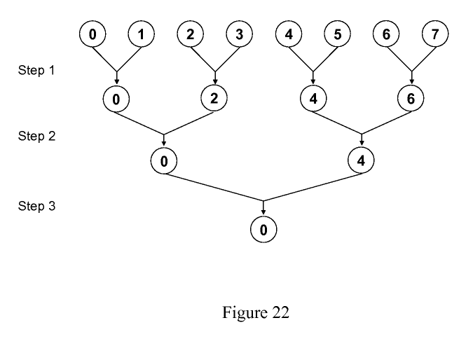

After full-scan searching, slaves calculate statistics, in step 1220 of FIG. 21, and discard insignificant matches in their result structure, spec_matchlist. The master waits until all slaves finish. The master then gathers all search results from all slaves into its result structure, spec_matchlsit. A modified MPI_Gather algorithm is used in the MS/MS database search program and is illustrated in FIG. 22.

As shown in FIG. 22, the master, process 0, gathers data from all slaves. Data from slaves are not required to be in the same size.

An overall statistics process is done by the master and the master distributes matches to itself and all slaves evenly.

Finally, in step 1240 of FIG. 21, the master and slaves do fine searching and create report files at the same time for the matches they are assigned.

The Message Passing Interface (MPI) MS/MS database search program was tested using a dataset containing 419 MS/MS spectra. The dataset was searched against human database with seven modifications and the number of peptides created from the database is 84,549,203. The search was performed on a Linux cluster with 512 900 MHz HP Itanium processors. As shown in FIG. 15, the speed of MPI MS/MS database search program is nearly proportional to the number of processors. The search time on a personal computer (PC) is typically shorter than data acquisition time (<two hours) for searched of the human protein database (NCBInr) with a modest number of modifications (<three). The parallelized MS/MS database search program provides near linear speed scaling and can search a data set with 4000 spectra against the human protein database with ten variable modifications in under ten minutes utilizing twenty CPUs on a 1.4 GHZ HP Itanium cluster. The searching speed was 0.9 million peptides per second when eighty processors were used.

Classification of Disulfide Bonds and Disulfide-L inked Peptides

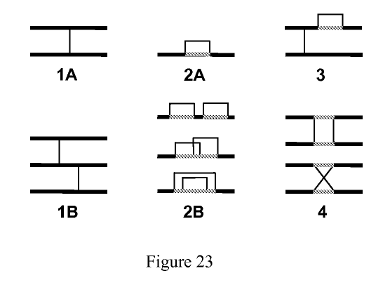

In MassMatrix, disulfide bonds in peptides were classified into two types: inter-chain disulfide bonds and intra-chain disulfide bonds. Peptides with more than 2 disulfide bonds are difficult to characterize by tandem MS due to poor fragmentation and size. Therefore, only peptides with up to 2 disulfide bonds were considered in MassMatrix. However, models for peptides with more than 2 disulfide bonds may be included in MassMatrix as needed. Peptides with up to 2 disulfide bonds were classified into 4 types as shown in FIG. 23. Type 1 peptides only have inter-chain disulfide bonds. Type 2 peptides only have intra-chain disulfide bonds. Type 3 peptides are hybrids with both inter- and intra-chain disulfide bonds. Type 4 peptides have circular chains. Peptides with only 1 disulfide bond fall into the classes: 1A and 2A. Peptides with 2 disulfide bonds include types: 1B, 2B, 3 and 4.

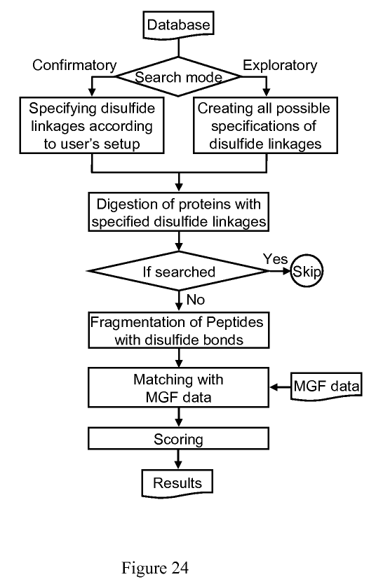

A flow chart for the disulfide search algorithm in MassMatrix is shown in FIG. 24. MassMatrix has two disulfide search modes: exploratory and confirmatory. In the exploratory search mode, all cysteine residues in the protein sequences are considered to be variable disulfide bonding sites, i.e. all cysteine may or may not form disulfide bonds. During searching, MassMatrix will generate all possible combinations of disulfide bonds by assuming that any two cysteine residues is capable of forming a disulfide bond. Consider a protein with n cysteine residues. During exploratory search mode, MassMatrix will generate

$${C}_{n}^{2} = \frac{n ( n - 1 )}{2}$$

possible combinations of single disulfide bond for the protein (n=number of cysteine residues). In the confirmatory search mode, disulfide bonds are specified in the sequence by input of a custom database. In the special .BAS MassMatrix databases, disulfide bonds are coded as “C($i)”, where is the index number of the specified disulfide bond. Each bond has two related disulfide bonding sites. The protein is digested in silico based on the specified proteolysis reagent with consideration given to the presence of disulfide bonds generated from either the exploratory search mode or as input by the user in confirmatory search mode. These hypothetical peptides are then fragmented using the appropriate fragmentation model and scored against the experimental tandem MS data.

In MassMatrix's Collision Induced Dissociation fragmentation model, disulfide bonds are not cleaved by collisional activation. Disulfide-linked peptide may have multiple chains that are connected to each other. Each chain may undergo fragmentation independently including neutral losses of H₂O and NH₃ due to residues with hydroxyl and amino groups respectively. Only product ions created from the rupture of a single bond were considered (thus internal fragments were not searched). Therefore, when one chain undergoes fragmentation to create product ions, other chain(s), if any, will be intact and considered as a modification to the cysteine on the first chain.

Type 1A peptides are the most preferred configuration for sequencing by tandem mass spectrometry. Type 1A peptide fragmentation produces a set of product ions for each peptide chain allowing for excellent sequence coverage. The same is true for type 1B given that the peptide chains are of reasonable length so that the precursor and product ions can be detected. Other peptide types can suffer from lower sequence coverage due to blind spots in the product ion series. For example, fragmentation of type 2 and 3 peptides that occurs in the sequence connecting the two linked sulfides all results in the same product ion and thus no useful sequence information. We refer to this region of the peptide (gray area in FIG. 23) as the blind spot. Similarly, for type 4 peptides fragmentation between the two inter-chain disulfide bonds on the same chain does not create any signature product ions. Resulting tandem MS spectra for type 2, 3 and 4 peptides will be missing product ions for these blind areas. At present the best strategy is to use a protease to cut these peptide sequences and convert them to type 1A. Alternatively we are examining the use of data dependent MS³ to sequence these blind areas.

The scoring algorithm involved in the disulfide bond search is the same as that used for peptides without disulfide bonds. MassMatrix contains three independent scoring models, including two statistical models and a descriptive model. The two statistical scores, pp and pp2, are the negative common logarithm of the likelihood that the peptide match is random. The pp value is the major standard used in MassMatrix and the pp2 value is a supplementary score. Peptide matches with either pp or pp2 value greater than 6 were considered to be statistically significant.

Validation with Disulfide-Linked Peptide Standards

The search algorithm was first tested by searching CID MS/MS data of two peptide standards and an insulin digest with known disulfide bonds. The peptide standards were type 2A and 2B peptides. Insulin consists of two chains connected by two inter-chain disulfide bonds and one chain also has an intra-chain disulfide bond. Digestion of insulin by a combination of trypsin and chymotrypsin creates two types of disulfide-linked peptides: type 1A and type 3 peptides. Each of these four types was successfully identified by searching their tandem MS data with the MassMatrix search engine. A listing of the peptide matches is provided in Table 7. Four types of standard disulfide-linked peptides identified in MassMatrix. * The match for type 2B standard peptide is considered significant by MassMatrix due to its significant pp2 value which is 9.5.

TABLE 7

| | | High pp | |
|---|---|---|---|
| Peptides | Type | value | Source |
| 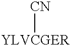 | 1A | 17.0 | Digest of Insulin |
| 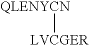 | 1A | 18.8 | Digest of Insulin |
| 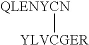 | 1A | 12.2 | Digest of Insulin |
| 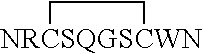 | 2A | 8.9 | Peptide standard 1 |
| 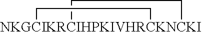 | 2B | 3.4* | Peptide standard 2 |
| 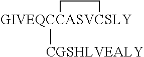 | 3 | 11.2 | Digest of Insulin |

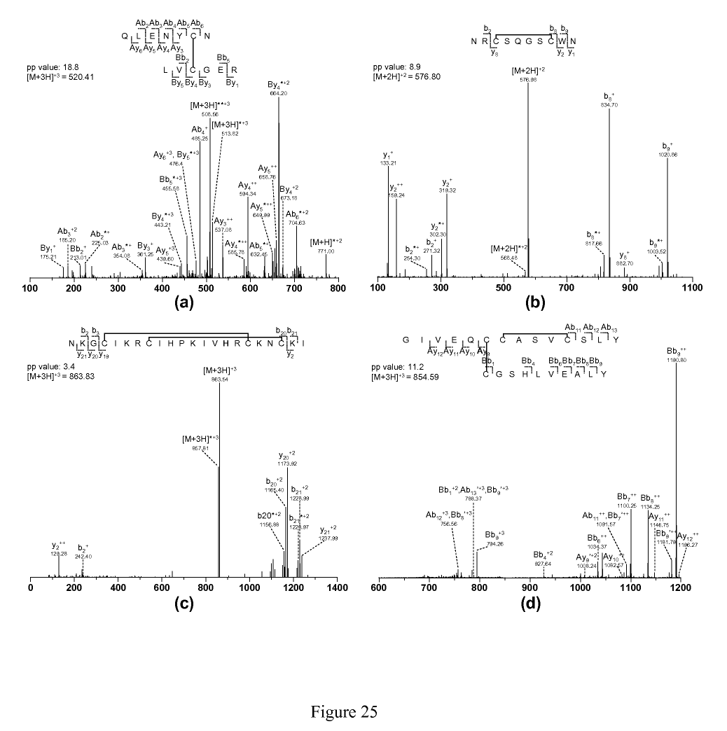

Because type 1A peptides have a higher potential to return a complete set of signature product ions created from fragmentation, they normally hive relatively higher pp values (FIG. 25a, Ions with neutral loss of small molecules are labeled by * (loss of ammonia) and ′ (loss of water)). Type 2 peptides (FIGS. 25b & 25c) contain blind spots and are more difficult to identify in tandem MS database search especially when a large part of their sequences is contained in the blind spot. This is manifest in lower pp scores as evident in the search of the type 2B peptide (FIG. 25c) listed in the Table 7. Fragmentation that occurs in the blind spot creates product ions with the same mass as the precursor ion. Therefore, we normally observe an abundant peak with the same mass-to-charge ratio (m/z) as the precursor ion in the tandem MS spectra for type 2 peptides as shown in FIGS. 25b & 25c. Wₑ are exploring the option of additional MS³ experiment to further map and identify these peptides. Type 3 peptides are hybrid where one chain has an intra-chain disulfide bond and a blind area linked via an inter-chain disulfide to a separate peptide (FIG. 25d). Because these peptide partially resemble type 1A, they can still be identified with significant scores in MassMatrix (FIG. 25d).

Validation with Bovine Pancreatic Ribonuclease A (RNaseA)

The tandem MS data sets from the digests of RNaseA by a combination of chymotrypsin and trypsin under basic (pH=8.0) and acidic (pH=6.0) conditions were searched against the RNaseA sequence using either the confirmatory or exploratory search modes. For the exploratory search, decoy sequences were added to a limited database that contained RNaseA, the reversed RNaseA sequence and 20 randomized RNaseA sequences. The summary of the searches for the data set from the digest under pH=8.0 are listed in Table 8. The summary of the three searches for the tandem MS data sets from RNaseA.

TABLE 8

| | | | Theoretical peptides | Search |
|---|---|---|---|---|
| | Database | Search mode | calculated | time |
| 1 | RNaseA | Confirmatory | 971 | 23 sec |
| 2 | RNaseA | Exploratory | 9,648 | 28 sec |
| 3 | RNaseA and | Exploratory | 232,481 | 62 sec |
| | decoys | | | |

Because the search space for the decoy sequences was much larger than that for the true RNaseA sequence peptide matches to the true sequence were considered true positives (TPs) and those from decoy sequences were considered false positives (FPs). The true positive peptide matches from RNaseA were further manually validated by two experts independently and those that passed both manual validations were used to map the disulfide bonds of RNaseA.

Confirmatory Search for Native Disulfide Bonds. Four native disulfide bonds in RNaseA, C26-C84, C40-C95, C58-C110 and C65-C72 were specified in the confirmatory search. The summary report of the four native disulfide bonds is shown in Table 9. Native disulfide-linked peptides in digest of RNaseA under basic and acidic conditions identified in the confirmatory search. * Peptides with intra-chain disulfide bond.

TABLE 9

| Digestion | Disulfide | number of | pp value | disulfide-linked |
|---|---|---|---|---|
| condition | bond | matches | sum/high | peptides |
| pH = 8.0 | C26-C84 | 41 | 554.9/21.9 | [26-29]-S-S-[80-85] |
| | | | | [26-30]-S-S-[80-85] |
| | | | | [26-31]-S-S-[80-85] |
| | | | | [26-31]-S-S-[77-85] |
| | C40-C95 | 74 | 629.3/18.9 | [40-46]-S-S-[93-97] |
| | | | | [40-46]-S-S-[92-97] |
| | | | | [40-46]-S-S-[92-98] |
| | C58-C110 | 10 | 112.7/17.2 | [47-61]-S-S-[105-115] |
| | C65-C72 | 58 | 807.6/32.9 | [62-73]* |
| | | | | [62-76]* |
| | | | | [62-66]-S-S-[67-73] |
| | | | | [62-66]-S-S-[67-76] |
| pH = 6.0 | C26-C84 | 32 | 431.6/20.5 | [26-29]-S-S-[80-85] |
| | | | | [26-30]-S-S-[80-85] |
| | | | | [26-31]-S-S-[80-85] |
| | C40-C95 | 47 | 386.4/15.4 | [40-46]-S-S-[93-97] |
| | | | | [40-46]-S-S-[92-97] |
| | | | | [40-46]-S-S-[92-98] |
| | | | | [38-46]-S-S-[92-97] |
| | C58-C110 | 11 | 129.8/17.5 | [52-61]-S-S-[105-115] |
| | | | | [47-61]-S-S-[105-115] |
| | C65-C72 | 6 | 79.3/21.9 | [62-73]* |
| | | | | [62-66]-S-S-[67-73] |
| | | | | [62-66]-S-S-[67-76] |

For the RNaseA digests under both basic and acidic conditions, each native disulfide bond was confirmed by multiple validated true peptide matches with significant statistical scores. More native disulfide-linked peptides and higher total statistical scores for native disulfide bonds were observed in basic digestion due to the fact that the optimal pH for trypsin digestion is pH ˜8.0. Therefore, all four native bonds reported by literature were confirmed with a very high confidence in our tandem MS experiment and subsequent database search performed in MassMatrix.

Exploratory Search for Native and Normative Disulfide Bonds. In the exploratory mode, MassMatrix searched the complete combination of all possible disulfide bonds in the protein with no assumptions for biological feasibility provided by the model. All native disulfide-linked peptides that were seen in the confirmatory search were also seen in the exploratory search for both basic and acidic digests.

For the RNaseA digest under basic condition, 18 normative disulfide-linked peptides were observed with multiple validated true positive matches for each peptide in the exploratory search (Table 10-Normative disulfide-linked peptides in digest of RNaseA under basic and acidic conditions identified in the exploratory search).

TABLE 10

| Digestion | Disulfide | number of | pp value | disulfide-linked |
|---|---|---|---|---|
| condition | bond | matches | sum/high | peptides |
| pH = 8.0 | C26-40 | 7 | 69.3/12.6 | [26-31]-S-S-[40-46] |
| | C26-C95 | 8 | 89.7/13.5 | [26-31]-S-S-[92-97] |
| | C26-C110 | 8 | 32.4/6.3 | [26-31]-S-S-[105-115] |
| | C40-C58 | 2 | 13.1/6.7 | [40-46]-S-S-[47-61] |
| | C40-C65 | 8 | 107.8/18.5 | [40-46]-S-S-[62-66] |
| | C40-C84 | 3 | 27.8/11.7 | [40-46]-S-S-[80-85] |
| | C40-C110 | 10 | 51.8/6.3 | [40-46]-S-S-[105-115] |
| | C58-C65 | 2 | 10.4/5.2 | [47-61]-S-S-[62-66] |
| | C58-C95 | 2 | 25.7/13.7 | [47-61]-S-S-[92-97] |
| | C65-C84 | 3 | 48.6/16.7 | [62-66]-S-S-[80-85] |
| | C65-C95 | 5 | 56.4/13.7 | [62-66]-S-S-[92-97] |
| | C65-C110 | 4 | 36.3/11.0 | [62-66]-S-S-[105-115] |
| | C84-C95 | 28 | 286.2/16.4 | [80-85]-S-S-[93-98] |
| | | | | [80-85]-S-S-[92-97] |
| | | | | [80-85]-S-S-[92-98] |
| | C84-C110 | 4 | 38.1/11.4 | [80-85]-S-S-[105-115] |
| | C95-C110 | 4 | 11.6/3.5 | [92-97]-S-S-[105-115] |
| | | | | [92-98]-S-S-[105-115] |
| pH = 6.0 | C84-C95 | 8 | 121.4/20.0 | [80-85]-S-S-[92-97] |

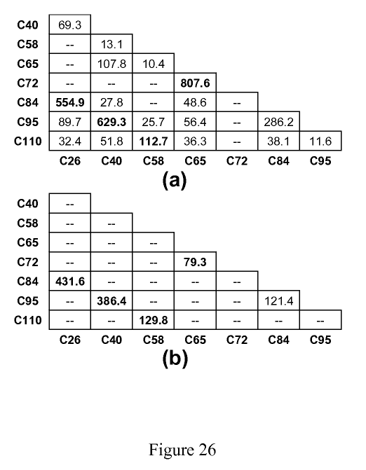

These peptides indicate that there are 15 normative disulfide bonds in the RNaseA. The summary of disulfide bond mapping in the RNaseA digests under basic is shown in FIG. 26a. Out of all 28 possible disulfide bonds formed by the eight cysteine residues in the RNaseA, 19 were identified in MassMatrix. These normative disulfide bonds are likely due to disulfide bond interchange during the sample processing and protein digestion. Confidence of the identification for each bond is indicated by the sum of pp values of peptide matches with the bond. Native disulfide bonds are bolded.

Compared with the RNaseA digest under basic condition, similar results for the four native disulfide bonds were observed in the RNaseA digest under acidic condition as shown in Table 9. However, only one validated normative disulfide bond (C84-C95) was observed FIG. 26b and Table 10. This could be due to the fact that the disulfide bond interchange was minimized by dilute acid. These observations were further supported by blocking disulfide interchange by treatment with iodoacetamide and enhancing interchange by heating during digestion.

Validation of MassMatrix Scoring Model for Disulfide-Linked Peptides

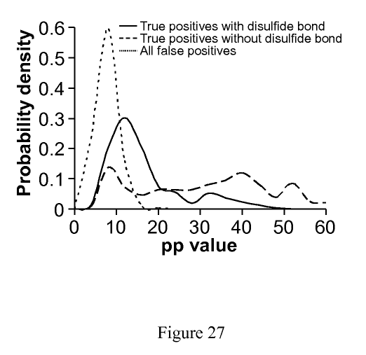

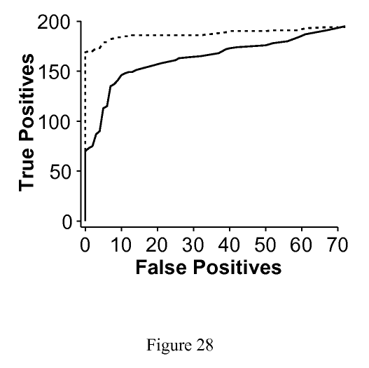

The scoring model for disulfide-linked peptides was validated by examination of the distributions of pp values for true positives and false positives (FIG. 27) and receiver operating characteristic (ROC) analysis (FIG. 28) for the data set from the digest under pH=8.0. True and false positives were determined from the searches of the RNaseA data sets with decoy sequences. The peptide matches from RNaseA in the search with dominating decoy sequences were considered true positives (TPs) and those from decoy sequences were considered false positives (FPs).

It can be seen that the scoring model was able to discriminate TPs from FPs at a low mass accuracy of 1.0 Da, although there is some overlap between the distributions of pp value for TPs and FPs. ROC analysis also indicates that the scoring performs well for both peptides without peptide bonds and disulfide-linked peptides. The ROC curve for peptides with disulfide indicates that the scoring model is able to identify the majority of TPs with disulfide bonds at a low rate of FPs (<10%).

Disulfide-Linked Peptides vs Peptides without Disulfide Bonds

Disulfide-linked peptides tend to have smaller pp values than those without disulfide bonds in MassMatrix as shown in FIG. 27, and MassMatrix has better sensitivity and specificity for peptides without disulfide bonds than for disulfide-linked peptides as indicated in the ROC analysis as shown in FIG. 28. False positives include both disulfide-linked false peptides and false peptides without disulfide bonds. In ROC curves, a value to the top indicates higher sensitivity and a value to the left indicates higher specificity. This is because 1) peptides with intra-chain disulfide bonds often do not contain the complete set of signature product ions due to blind spots and 2) peptides with inter-chain disulfide have product ions clustered at low and high m/z. However, peptides without any disulfide bonds have product ions spread across the whole mass range resulting in a better signature.

A new database search algorithm has been developed to identify disulfide-linked peptides in tandem MS data sets. The algorithm was based on the statistical scoring model in MassMatrix and has been incorporated in MassMatrix to enable the program to map disulfide bonds form tandem MS data. The algorithm was tested on peptide and protein standards with known disulfide bonds. All disulfide bonds in the standard set were identified with high statistical scores by MassMatrix. The algorithm was further tested on bovine pancreatic ribonuclease A (RNaseA). The 4 native disulfide bonds in RNaseA were detected by MassMatrix with multiple validated peptide matches for each bond and high statistical scores. 15 normative disulfide bonds were also observed in the digest under basic conditions (pH=8.0) which was mainly due to random disulfide bond interchange during digestion. After minimizing the disulfide bond interchange by dilute acid (pH=6.0) during digestion, only one normative disulfide bond was observed. The distribution of statistical scores for true and false positives and ROC analysis indicate that the algorithm is capable to discriminate TPs with disulfide bonds from FPs and the program can identify majority of the TPs with disulfide bonds at a low false rate (<10%).

All peptide and protein standards were purchased from Sigma-Aldrich (St. Louis, Mo.). The protein standards were digested by a combination of trypsin (Promega, Madison Wis.) and chymotrypsin (Roche, Switzerland) following a protocol similar to W. K. Ressell et al. Briefly, protein standards were digested in 25 mM ammonium bicarbonate solution (pH=8.0). In order to minimize/block the disulfide bond interchange, protein standards were also digested in 25 mM phosphate Buffer (pH=6.0) or 25 mM ammonium bicarbonate with 10 mM iodoacetamide (final concentration). Enzymes were used in 25:1 ratio (substrate:enzyme) and the mixture was incubated at 37° C. for overnight.

Capillary-liquid chromatography-nanospray tandem mass spectrometry (Nano-LC/MS/MS) was performed on a Thermo Finnigan LTQ mass spectrometer equipped with a nanospray source operated in positive ion mode. The LC system was an UltiMate™ Plus (Dionex, Sunnyvale, Calif.) with a FAMOS autosampler and SWITCHOS column switcher. Solvent A contained water with 50 mM acetic acid and solvent B contained acetonitrile. Five microliters of each sample was injected onto the trapping column (LC-Packings A Dionex Co, Sunnyvale, Calif.), and washed with Solvent A. The injector port was switched to inject and the peptides were eluted off of the trap onto a 5 cm 75 μm ID ProteoPep II C18 packed nanospray tip (New Objective, Inc. Woburn, Mass.). Peptides were eluted into the LTQ system using a gradient of 2-80% B over 30 minutes, with a flow rate of 300 nl/min. The total run time was 58 minutes. The scan sequence of the mass spectrometer was programmed to perform a full scan followed by 10 data-dependent MS/MS scans of the most abundant peaks in the spectrum. Dynamic exclusion was used to exclude multiple MS/MS of the same peptide.

All spectra that were not determined as singly charged were searched as both doubly and triply charged ions. Peptide sequences with a length from 6 to 50 amino acid (AA) residues, missed cleavage sites of up to 4, and charges of +1, +2, and +3 were searched. For spectra with multiple matches, the highest scoring match was used. The program was tested with peptide and protein standards and with the well characterized protein, RNaseA, which contains four known native disulfide bonds.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A method of matching tandem mass spectra data simultaneously and automatically to theoretical peptide sequences derived from a biological sequence database, the method comprising:
digesting a biological sequence into smaller unit sequences as specified by a user;
skipping processing of redundant smaller unit sequences;
modifying and fragmenting the smaller unit sequences to create a theoretical spectrum for the smaller unit sequence;
matching the modified and fragmented smaller unit sequences against tandem mass spectra data within specified tolerances;
calculating four scores for each potential match, wherein the four scores comprise three statistical derived scores and one empirically derived score;
discarding potential matches below a critical threshold, wherein the critical threshold is based on the calculated scores; and
matching remaining smaller unit matches with corresponding biological sequences.

2. The method of claim 1, wherein the biological sequence comprises a protein, isotopes, DNA, RNA, carbohydrate side-chains, or combinations thereof.

3. The method of claim 1, further comprising:
filtering the tandem mass spectra data to reduce noise prior to matching against the modified and fragmented smaller unit sequences.

4. The method of claim 1, further comprising:
using a message passing interface schema to matching the theoretical spectrum against the tandem mass spectra data.

5. The method of claim 1, wherein the three statistical derived scores are the negative common logarithm of the likelihood that potential smaller unit match is random.

6. The method of claim 5, wherein the likelihood that potential smaller unit match is based on mass accuracy.

7. The method of claim 1, wherein among the three statistical derived scores, two statistical derived scores are sensitive to the accuracy of the mass spectrometer.

8. The method of claim 1, further comprising:
calculating Monte Carlo based scores after discarding potential matches.

9. The method of claim 1, further comprising:
outputting the results of matching the remaining smaller unit matches with the corresponding biological sequences.

10. The method of claim 9, wherein the output is in html format, XML format or combinations thereof.

11. The method of claim 1, wherein the method is portable.

12. A method of matching tandem mass spectra data simultaneously and automatically to theoretical peptide sequences derived from a protein database, the method comprising:
digesting a protein sequence into peptides sequences;
modifying and fragmenting the peptide sequences;
matching the modified and fragmented peptide sequences against tandem mass spectra data;
calculating three scores for each potential match, wherein the four scores comprise three statistical derived scores and one empirically derived score;
discarding potential matches below a critical threshold, wherein the critical threshold is based on the calculated scores;
refining the potential matches by calculating Monte Carlo Simulation based scores; and
matching remaining peptide matches with corresponding protein sequences.

13. The method of claim 12, wherein one of the three statistical derived scores is based on the number of matched product ions, the second of the three statistical derived scores is based on number of sequence tags, and the third of the two statistical derived scores is based on total abundance of the matched product ions.

14. The method of claim 12, wherein the two of the two statistical derived scores based on the number of matched product ions and based on sequence tags are the major standard score used and the other of the two statistical derived scores based on total abundance of the matched product ions is a supplementary score.

15. The method of claim 12, further comprising:
automatically searching for disulfide linkage bonds.

16. The method of claim 12, further comprising:
predicting retention time by calculating a statistical score based on hydrophobicity of the peptide sequence.

17. A method of searching tandem mass spectra data simultaneously and automatically to theoretical peptide sequences derived from a protein database for disulfide bonds, the method comprising:
specifying type of disulfide bonds for searching;
digesting a protein sequence with specified disulfide linkages into peptides sequences;
skipping processing of redundant smaller unit sequences;
fragmenting the peptide sequences with specified disulfide bonds;
matching the fragmented peptide sequences against tandem mass spectra data;
calculating four scores for each potential match, wherein the four scores comprise three statistical derived scores and one empirically derived score;
discarding potential matches below a critical threshold, wherein the critical threshold is based on the calculated scores; and
matching remaining peptide matches with corresponding protein sequences.

18. The method of claim 17, wherein the types of disulfide bond searching include exploratory and confirmatory.

19. The method of claim 18, wherein exploratory searching considers cysteine residues in the protein sequences to be variable disulfide bonding sites.

20. The method of claim 18, wherein disulfide bonds are specified in the protein sequence by input of a custom database in confirmatory searching.

21. A method of matching tandem mass spectra data simultaneously and automatically to theoretical peptide sequences derived from a protein database, the method comprising:
determining a probability based peptide score by incorporating mass spectra accuracy into scoring potential peptide and protein matches; and
correlating a probability that a match is a random occurrence.
